(19) United States
(12) Patent Application Publication
(10) Pub. No.: US 2023/0210688 A1
(43) Pub. Date: Jul. 6, 2023

(54) WEARABLE HEAT TRANSFER DEVICES AND ASSOCIATED SYSTEMS AND METHODS

(71) Applicant: BlueXthermal, Inc., Cambridge, MA (US)

(72) Inventors: Reza MONAZAMI, Boston, MA (US); Sahar JAHANI, Boston, MA (US); Nicholas Keith ANSELMO, Yorktown, VA (US)

(21) Appl. No.: 18/149,625

(22) Filed: Jan. 3, 2023

Related U.S. Application Data
(60) Provisional application No. 63/266,401, filed on Jan. 4, 2022.

(51) Int. Cl. A61F 7/00

(52) U.S. Cl. CPC A61F 7/0085; A61F 2007/0056

(57) ABSTRACT

Thermal management devices and associated systems and methods are disclosed herein. In some embodiments, a representative device can comprise (i) thermoelectric components (TECs) each including a first side configured to be operated at a desired temperature and a second side opposite the first side, and (ii) a heat transfer system including an array of fluid distribution networks, an inlet passage coupled to the fluid distribution networks, and an outlet passage coupled to the fluid distribution networks. In operation, a working fluid flows through the fluid distribution networks from the inlet passage to the outlet passage, and absorbs heat from the fluid distribution networks. The inlet and outlet passages can be fluidically coupled to individual fluid distribution networks such that pressure drop and/or temperature drop of the working fluid across the individual fluid distribution networks is about the same as one another.

### CROSS-REFERENCE TO RELATED APPLICATIONS

The present application claims the benefit of priority to U.S. Provisional Patent Application No. 63/266,401, filed Jan. 4, 2022, and is related to U.S. patent application Ser. No. 17/183,313, titled WEARABLE HEAT TRANSFER DEVICES AND ASSOCIATED SYSTEMS AND METHODS, filed Feb. 23, 2021, and to U.S. patent application Ser. No. 18/149,574, titled OCULAR REGION HEAT TRANSFER DEVICES AND ASSOCIATED SYSTEMS AND METHODS, filed Jan. 3, 2023, the disclosures of which are incorporated herein by reference in their entireties.

### TECHNICAL FIELD

This present disclosure relates to heat transfer devices configured to be worn by a user, and associated systems and methods.

### BACKGROUND

Many types of devices and systems produce significant heat fluxes and there is a growing demand for advanced and efficient systems capable of extracting and dissipating such heat fluxes to keep temperatures within acceptable operating ranges. Many wearable devices, for example, dissipate heat from a target area to reduce pain or swelling, change tissue structures (e.g., reduce adipose tissue and treat skin conditions), or mitigate localized heating of tissue caused by other procedures (e.g., laser treatments). Wearable devices are desirably lightweight and portable, but this presents a challenge for dissipating the significant heat fluxes required in many applications. As a result, a significant gap exists between the required heat transfer performance for many applications and the heat transfer performance of existing devices and systems. For example, current heat transfer systems are often large and heavy to provide adequate heating or cooling for controlling swelling and other post-surgical applications. Therefore, such systems are cumbersome and can be uncomfortable in a wearable device, and they are often too large to work with the complex contours of certain anatomical features. Moreover, heat treatment applications for more sensitive areas, such as under-eye tissue, are limited and unable to provide consistent active cooling treatment for the necessary time duration, which is often needed to freeze and kill corresponding fat cells. As a result, a need exists for an improved wearable heat transfer device.

### BRIEF DESCRIPTION OF THE DRAWINGS

Features, aspects, and advantages of the presently disclosed technology may be better understood with regard to the following drawings.

FIG. 1 is a partially schematic cross-sectional view of heat a transfer device around a portion of a mammal, in accordance with embodiments of the present technology.

FIG. 2 is a partially schematic enlarged cross-sectional view of a portion of the heat transfer device shown in FIG. 1.

FIG. 3A is a partially schematic cross-sectional isometric view of a portion of the heat transfer device shown in FIG. 2.

FIG. 3B is a partially schematic cross-sectional isometric view of a heat transfer structure, in accordance with embodiments of the present technology.

FIG. 4 is a partially schematic top view of the heat transfer device of FIG. 1.

FIG. 5A is a partially schematic top view of a heat transfer system of a wearable heat transfer device, in accordance with embodiments of the present technology.

FIG. 5B is a partially schematic cross-sectional side view of the heat transfer system of FIG. 5A.

FIG. 6 is a partially schematic top view of a heat transfer system of a heat transfer device, in accordance with embodiments of the present technology.

FIG. 7 is a partially schematic bottom view a heat transfer device with thermally conductive members, in accordance with embodiments of the present technology.

FIG. 8A is a partially schematic bottom view of an expandable heat transfer device in an unexpanded state and including thermally conductive members, in accordance with embodiments of the present technology.

FIG. 8B is a partially schematic bottom view of the heat transfer device of FIG. 8A in an expanded state.

FIG. 9 is a partially schematic bottom view of a heat transfer device expandable in multiple directions and including thermally conductive members, in accordance with embodiments of the present technology.

FIG. 10 is a schematic exploded isometric view of a heat transfer device including thermally conductive members, in accordance with embodiments of the present technology.

FIG. 11 is a schematic exploded isometric view of a heat transfer device with thermally conductive members, in accordance with embodiments of the present technology.

FIG. 12 is a schematic top view of an arrangement of thermoelectric components of a heat transfer device, in accordance with embodiments of the present technology.

FIG. 13 is a schematic top view of an arrangement of thermoelectric components of a heat transfer device, in accordance with embodiments of the present technology.

FIG. 14 is a schematic top view of arrangements of thermoelectric components of a heat transfer device, in accordance with embodiments of the present technology.

FIG. 15A is a partially schematic view of a heat transfer device being worn by a human, in accordance with embodiments of the present technology.

FIGS. 15B-15D are partially schematic views of a heat transfer system including the heat transfer device of FIG. 15A, in accordance with embodiments of the present technology.

FIGS. 16-24 are partially schematic views of heat transfer devices being worn by a human at various target areas, in accordance with embodiments of the present technology.

FIG. 25 is a schematic block diagram illustrating a system incorporating a heat transfer device, in accordance with embodiments of the present technology.

FIG. 26 is a flow diagram illustrating a method for treating a human via a heat transfer device, in accordance with embodiments of the present technology.

FIG. 27 is a flow diagram illustrating a method for controlling the temperature of a target area of a human with a heat transfer device, in accordance with embodiments of the present technology.

A person skilled in the relevant art will understand that the features shown in the drawings are for purposes of illustration, and variations, including different and/or additional features and arrangements thereof, are possible.

### DETAILED DESCRIPTION

### I. Overview

Many types of devices and systems produce significant heat fluxes and there is a growing demand for advanced and efficient systems capable of extracting and/or dissipating such heat fluxes to keep temperatures within acceptable operating ranges. Many wearable devices, for example, dissipate heat from a target area to reduce pain or swelling, change tissue structures (e.g., reduce adipose tissue and treat skin conditions), or mitigate localized heating of tissue caused by other procedures (e.g., laser treatments). Wearable devices are desirably lightweight and portable, but this presents a challenge for dissipating the significant heat fluxes required in many applications. As a result, a significant gap exists between the required heat transfer performance for many applications and the heat transfer performance of existing devices and systems. For example, current single-phase systems are often large and heavy to provide adequate heating or cooling for controlling swelling and other post-surgical applications. Therefore, such systems are cumbersome and can be uncomfortable in a wearable device, and they are often too large to work with the complex contours of certain anatomical features, including the knee, shoulder, ankle, leg, arm, back, head, neck, and/or elbow regions.

Sleeves with circulating coolant and ice/gel packs are currently the most prevalent wearable heat transfer devices for thermally treating a target tissue area, e.g., reducing the temperature of the tissue. For example, with fluid-circulated sleeves a cold fluid at a single temperature is circulated by a pump through a sleeve wrapped around a target area. The temperature of tissue in the target area drops as heat is conducted across the sleeve and absorbed by the circulated cold fluid. The heated fluid is returned to an ice bath, and the heat removed from the tissue is absorbed by the ice as it melts. Ice/gel packs work similarly by absorbing heat from the target area by warming the cold ice or gel within the pack.

Both of these wearable devices have significant shortcomings, including (i) the lack of temperature control at which the tissue is exposed or cooling therapy control relative to musculoskeletal or similar structures surrounding target tissue, (ii) a limited time period or capacity for cooling, (iii) an inability to receive continuous cooling therapy without adjusting or tending to the device, and (iv) a lack of flexibility of the device, e.g., due to the pressurized liquid flow and/or rigidness of the icepacks, therein causing an uncomfortable fit for the user. The lack of flexibility can further limit the amount of heat transfer between the device and user as the inflexible nature of the device prevents a conforming fit and/or optimal thermal contact between the device and user. As a result, current wearable devices are unable to adequately thermally treat the target area of a mammal and are generally ineffective in treating underlying conditions (e.g., pain, swelling, overheating, diminished blood perfusion, diminished nerve connectivity, stroke, etc.).

Embodiments of the present disclosure address at least some of the above-described issues by providing a thermal management device and system that, among other features, is safer, allows for better temperature control, and enables enhanced thermal contact between the device and the user/mammal by being flexible, lighter and thinner than current related devices. For example, as described herein, embodiments of the present disclosure can include (i) thermoelectric components arranged in an array and spaced apart from each other to be thermally coupled to a target of a mammal, (ii) a heat transfer system thermally coupled to the thermoelectric components. The heat transfer system can include a heat exchanger, fluid distribution networks, an inlet (and/or cold working fluid) passage fluidically coupled to the fluid distribution networks and configured to provide a working fluid to the fluid distribution networks, and an outlet (and/or hot working fluid) passage fluidically coupled to fluid distribution networks and configured to receive the working fluid from the fluid distribution networks. In some embodiments, the thermal management device and system include a flexible support unit coupled to the thermoelectric components and configured to compress against the target area of the mammal, such that the thermoelectric components are arranged to thermally threat the target area. The flexible support unit may include thermally conductive members positioned across two or more thermoelectric components and which distribute heat between thermoelectric components and the device. The thermoelectric components can each be individually, or as a group, controlled (e.g., set to a particular temperature) by a controller operably coupled thereto. As such, individual regions of the device can be set to different temperatures relative to other regions and can thus individually treat corresponding target areas of the mammal that the device is positioned on or around. When in a cooling mode, heat can flow from the target area to the thermoelectric components and to the heat transfer system. In doing so, embodiments of the present disclosure enable rapid and controlled cooling for treating certain underlying conditions such as pain, swelling, overheating, diminished blood perfusion, diminished nerve connectivity, and/or stroke, while mitigating damage to the epidermal and/or dermal tissues.

In some embodiments, the fluid distribution networks (also referred to herein as heat transfer structures) of the heat transfer system include a first fluid distribution network and a second fluid distribution network, in which the first fluid distribution network is coupled to a first area of the inlet passage and the second fluid distribution network is coupled to a second area of the inlet passage downstream of the first area of the inlet passage. In such embodiments, the first fluid distribution network can be coupled to a first area of the outlet passage and the second fluid distribution network is coupled to a second area of the outlet passage downstream of the first area of the outlet passage. Advantageously, such embodiments of the thermal management device enable the pressure drops of the working fluid across each of the fluid distribution networks to be about equal to one another or differ by less than a predetermined pressure threshold (e.g., within 10%). Stated differently, although the pressure of the working fluid at the inlets and outlets of the respective fluid distribution networks may be different, the pressure drops of the working fluid across each of the fluid distribution networks are about equal or differ by less than the predetermined pressure threshold. Additionally or alternatively, and as a result of the pressure drop profiles, the temperature drops across each of the fluid distribution networks is also about equal or differ by less than a predetermined temperature threshold (e.g., within 10%). This common temperature profile can help ensure the amount of heat removed from individual TECs across the device is about the same (e.g., on a proportional basis), thereby enabling the device to be more effective at removing or regulating heat from the target area and/or surrounding region.

In the Figures, identical reference numbers identify generally similar, and/or identical, elements. Many of the details, dimensions, and other features shown in the Figures are merely illustrative of particular embodiments of the disclosed technology. Accordingly, other embodiments can have other details, dimensions, and features without departing from the spirit or scope of the disclosure. In addition, those of ordinary skill in the art will appreciate that further embodiments of the various disclosed technologies can be practiced without several of the details described below.

### II. Heat Transfer Devices and Associated Systems and Methods

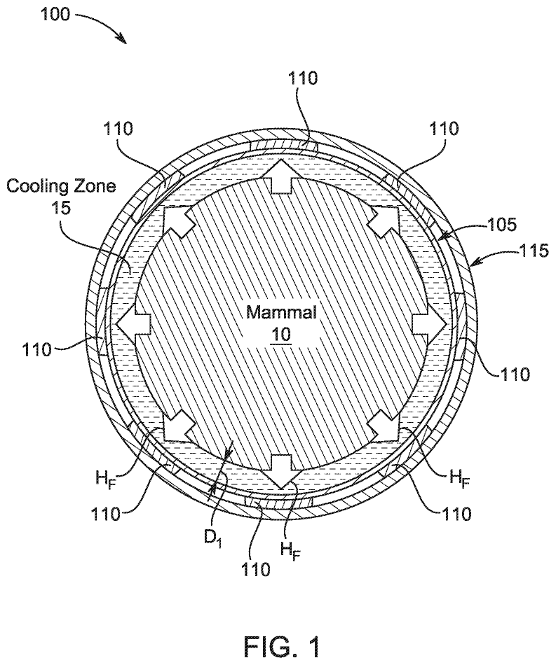

FIG. 1 is a partially schematic cross-sectional view of a heat transfer device 100 (“device 100”) around a portion of a mammal or human 10 (“human 10”)), in accordance with embodiments of the present technology. As shown in the illustrated embodiment, the device 100 includes (i) a flexible support unit 105 wrapped at least partially around a portion or target area (e.g., skin, tissue, arms, legs, knees, ankles, feet, shoulders, head, neck, face, elbows or any other body part area) of the human 10, (ii) thermoelectric components or modules 110 (“TECs 110”) over the flexible support unit 105 and thermally coupled to the human 10, and (iii) a heat transfer system or unit 115 thermally coupled to and configured to remove heat from the TECs 110. As described in additional detail herein, the heat transfer system 115 includes a single-phase heat transfer system and, in some embodiments, a two-phase heat transfer system. In operation, the TECs 110 can be set to a particular temperature and thus be configured to heat and/or cool the target area of the human 10. When the device 100 is in a cooling mode, for example, heat flow (H_{F}) transfers from the human 10 to the flexible support unit 105, to the individual TECs 110, and to the heat transfer system 115. As heat is removed from the human 10 in such a manner, a cooling zone 15 on the target area forms and can extend to a cooling depth (D₁) of the human. The depth (D₁) can be at least 1 millimeter (mm), 2 mm, 3 mm, 4 mm, or 5 mm, or within a range of 1-5 mm or any incremental range thereof (e.g., 1.5-3 mm). The cooling zone 15 can correspond to a heating zone when the device 100 is in a heating mode.

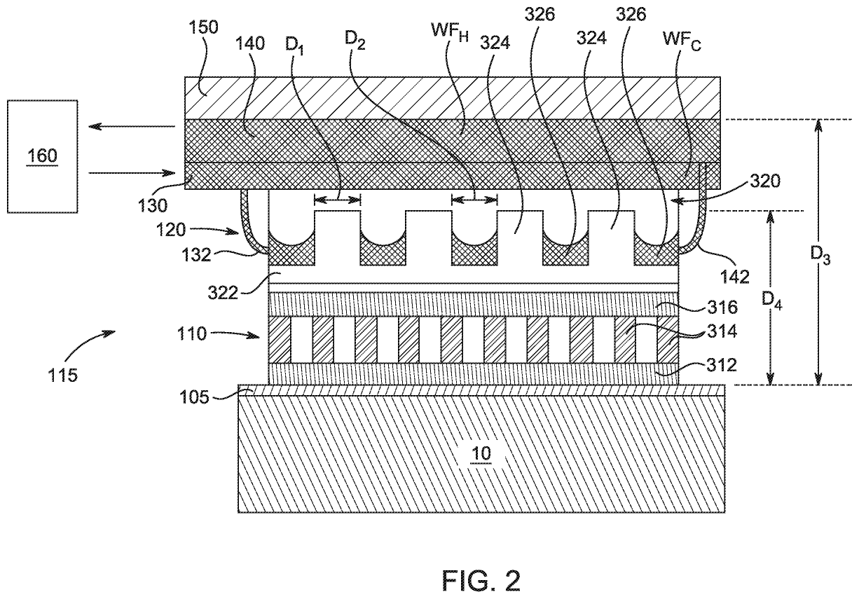

FIG. 2 is a partially schematic enlarged cross-sectional view of a portion of the device 100 of FIG. 1, in accordance with embodiments of the present technology. The heat transfer system 115 of FIGS. 1 and 2 is a single-phase heat transfer system. In some embodiments, the heat transfer system 115 can be a two-phase heat transfer system. The heat transfer system 115 can include (i) an array of fluid distribution networks or heat transfer structures 120 each thermally coupled to a corresponding one of the TECs 110, (ii) a cold working fluid passage 130 (e.g., an inlet working fluid passage) configured to provide a working fluid at a cold temperature (WF_{C}) to each of the heat transfer structures 120 (e.g., at a respective inlet 132 of each of the heat transfer structures 120), (iii) a heated working fluid passage 140 (e.g., an outlet working fluid passage) configured to receive the working fluid at a hot temperature (WF_{H}), higher than the temperature of the cold working fluid (WF_{C}), from each of the heat transfer structures 120 (e.g., at a respective outlet 142 of each of the heat transfer structures 120), (iv) an insulation member 150, and (v) a heat exchanger 160. In some embodiments, the insulation member 150 is omitted from the device 100. The heat exchanger 160 is configured to receive the heated working fluid (WF_{H}) from the heated working fluid passage 140, and provide the cold working fluid (WF_{C}) to the cold working fluid passage 130. In some embodiments, the heat exchanger 160 is passively air cooled or actively cooled with a cooling fluid provided via one or more pumps.

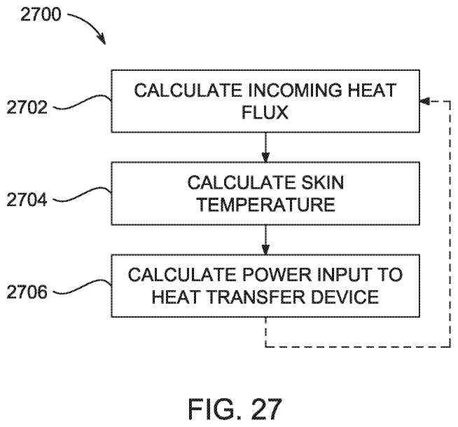

The TECs 110 can comprise a semiconductor-based electronic component configured to move heat from one side of the TEC 110 to a second opposing side of the TEC 110. The TECs 110 can provide precise, controllable, and/or localized temperature control at the interface between the target area and the device 100. As shown in FIG. 1, the TECs 110 are thermally coupled to the human 10 and can be set to a particular temperature and/or predetermined temperature profile (e.g., constant temperature profile, temperature cycle profile, and/or time based profiles) by a controller (e.g., the controller 2594; FIG. 27) to cool and/or heat the adjacent target area of the human 10. Setting the TECs 110 to a particular temperature can include providing an electrical current to the TECs 110 that corresponds to that temperature. For example, setting a first TEC 110 to a first temperature can include providing a first current level to the first TEC 110, and setting a second TEC 110 to a second temperature different than first temperature can include providing a second current level different than the first current to the second TEC 110. In doing so, the human 10 can experience desired therapy at only certain target areas.

As an example of how the TECs 110 may be operated, in some embodiments the first side of the TECs 110 facing the human 10 or the second side of the TECs 110 facing the heat transfer structures 120 can be set to a temperature within a range of 45° C. to −20° C. (e.g., 40° C., 35° C., 20° C., 5° C., 0° C., −5° C., −10° C., −15° C., etc.). In some embodiments, the TECs 110, either alone or in combination with the heat transfer structures 120, can be configured such that the second side of the TECs 110 is set or held at a first temperature or first temperature range and the first side of the TECs 110 are controlled to be cooled from normal surface body surface temperatures to a second temperature or second temperature range. In such embodiments, the second temperature or second temperature range can be more or less (e.g., 5° C., 10° C., 20° C., 30° C., or 40° C. more or less) than the first temperature or first temperature ranges. Additionally or alternatively, upon setting the temperature at the second side of the TECs 110, the first side of the TECs 110 can be configured to reach a desired temperature within a predetermined time, e.g., no more than 10 seconds, 20 seconds, 30 seconds, 40 seconds, or 60 seconds, or within a range of 10-60 second or any incremental range therebetween. As disclosed herein, operation of the TECs 110 may be based on a signal received from one or more sensors configured to detect temperature of the target area, the first side of the TEC 110, or the second side of the TEC 110.

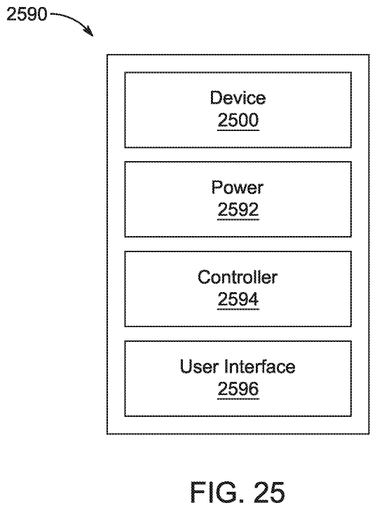

The TECs 110 can be placed in a heating mode, a cooling mode, or cycle between heating and cooling to control the temperature at the target area. Heat flow across an individual TEC 110 can be a function of temperature difference between its two side and/or the electric power input provide to the individual TEC 110 from a power source (e.g., power source 2592; FIG. 25). The mode and/or operation of the mode can be selected based on predetermined cycle times, temperature sensor feedback, and/or other parameters. When in the heating mode, the TECs 110 can provide heat to the target area of the human 10 (e.g., via the flexible support unit 105) by heating the first side of the TECs 110 which causes the second sides of the TECs 110 to cool. The heat transfer structures 120 can be controlled (e.g., turned off) to mitigate further cooling of the second side of the TECs 110. In some embodiments, the device 100 can further comprise additional resistive heaters that can be controlled via the controller and configured to heat the adjacent target area of the human 10.

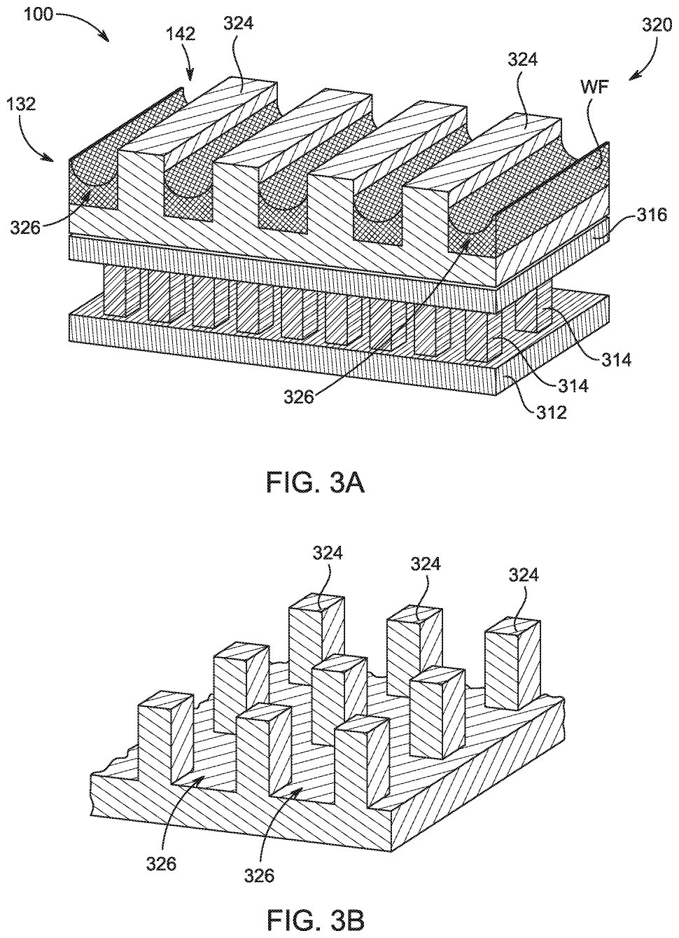

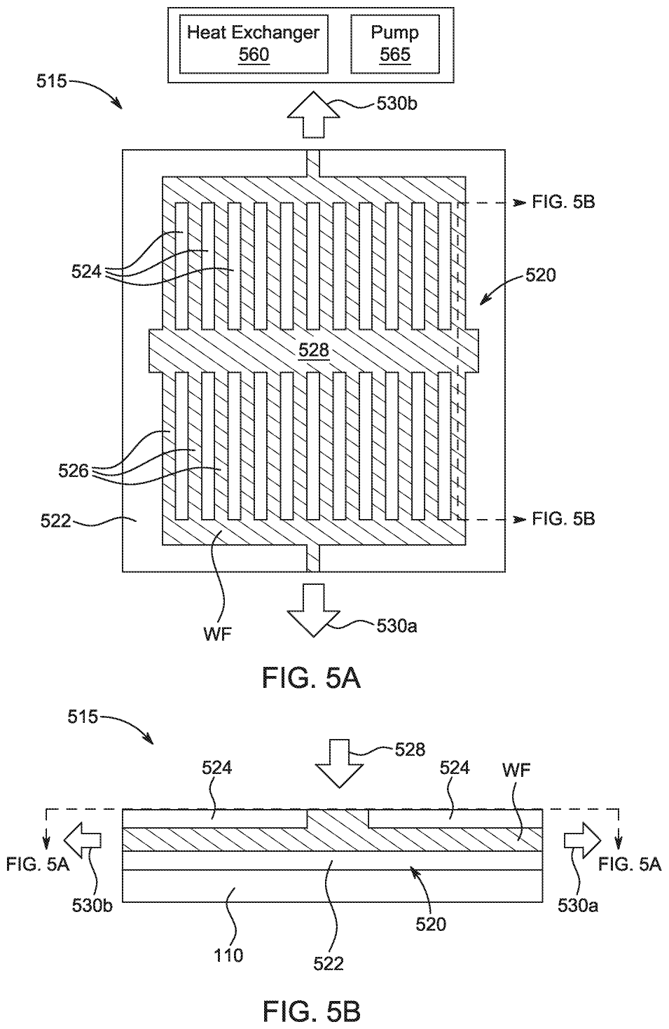

When in the cooling mode, the heat transfer structures 120 are configured to remove heat from hotter second sides of the TECs 110 and thereby enable the first sides of the TECs 110 to cool the adjacent target area of the human 10. As such, in the cooling mode heat flows from the target area of the human 10 in a radially outward direction to the TECs 110 and then to the heat transfer structures 120. As previously described, the TECs 110 can also cycle between the heating and cooling modes, which can enhance blood flow and perfusion to the target area. In some embodiments, parameters of the cooling and/or heating modes are based on or limited by safety considerations, such as a maximum heating or cooling temperature and/or maximum amount of heating or cooling time (e.g., 15 minutes, 20 minutes, etc.). Additional details regarding individual TECs 110 are provided herein (e.g., with reference to FIGS. 3 and 5).

The heat transfer system 115 can comprise a closed loop single-phase system, wherein flow of the working fluid through the heat transfer system 115 is driven by heat transferred from the TECs 110 to the individual heat transfer structures 120. In some embodiments, the heat transfer system can comprise a closed loop two-phase system, or include one or more pumps that drive flow of the working fluid through the heat transfer system 115. Additionally or alternatively, flow of the working fluid through the heat transfer system 115 is driven by gravity. For example, when driven by gravity, the heat exchanger 160 may be positioned physically above the other portions (e.g., the heat transfer structures 120) of the heat transfer system 115 such that gravity can provide enough force to circulate the working fluid to the transfer structures, where the temperature of the working fluid rises and returns to the heat exchanger 160 via the heated working fluid passage 140. Additionally or alternatively, flow of the working fluid through the heat transfer system 115 can be driven by capillary forces induced by microfeatures (e.g., pillars, pins, or walls) that form channels within chambers of the heat transfer structures 120 that drive the cold working fluid from inlets of the chambers toward the outlets of the chambers. Additionally or alternatively, in some embodiments the heat transfer system 115 can include a buffer vessel or reservoir configured to hold an excess amount of cold working fluid (WF_{C}), e.g., to ensure the supply of the cold working fluid (WF_{C}) can be continuously supplied. The buffer vessel can be particularly beneficial when the device 100 is operating at more extreme temperatures (e.g., 45° C., −20° C., etc.). In some embodiments the buffer vessel and the heat exchanger 160 may comprise a single integral unit.

The heat transfer structures 120 can each include a chamber 320, a base substrate or member 322 within the chamber 320, microfeatures 324 that protrude from the base member 322, and channels 326 formed between and defined by adjacent ones of the microfeatures 324. The heat transfer structures 120 can comprise an integral structure (e.g., a single component) and thus include a continuous surface extending along the base member 322 and the channels 326. As shown in FIG. 2, the working fluid (WF_{C} and WF_{H}) is positioned within the channels 326 and can form a meniscus, which is due in part to the properties of the working fluid and the microfeatures 324, or more particularly the heat of the microfeatures 324 and arrangement (e.g., spacing) of the microfeatures 324 relative to one another. Without being bound by theory, the meniscus can form a thin film portion at an interface with the adjacent microfeature walls that enhances efficient heat transfer from the TEC 110 to the heat transfer structure 120 and to the working fluid. In operation, the heat and/or arrangement of the microfeatures 324 induces capillary forces to the working fluid and causes the working fluid to move from the inlet region 132 at a first end of the chamber 320 to the outlet region 142 at a second opposing end of the chamber 320. Individual microfeatures 324 can have a lateral dimension (D₁) of 5 microns to 250 microns, and can be spaced apart from adjacent microfeatures 324 by a lateral dimension (D₂) of 5-1,000 microns.

As shown in the illustrated embodiment, the microfeatures 324 extend from the base member 322 away from the TECs 110. In other embodiments, the heat transfer structures 120 can be positioned in an opposite orientation with the base member 322 being adjacent the cold working fluid passage 130 or the insulation member 150 and the microfeatures extending from the base member 322 toward the TECs 110. In such embodiments, the heat transfer structures 120 includes a reservoir adjacent the TEC 110 and containing the cold working fluid (WF_{C}), and end portions of the microfeatures 324 are submerged within the cold working fluid (WF_{C}). In operation, the microfeatures 324 induce capillary forces on the cold working fluid (WF_{C}), increasing the temperature to generate heated working fluid (WF_{H}) that exits the chamber 320 through the heated working fluid passage 140.

The heat transfer structures 120 are each over one or more TECs 110. The cold working fluid passage 130 and the heated working fluid passage 140 are fluidically coupled to each of the heat transfer structures 120, or more particularly to the chambers 320 of the heat transfer structures 120. For example, for an individual heat transfer structure 120 the cold working fluid (WF_{C}) is supplied from the cold working fluid passage 130 to an inlet 132 (e.g., one of the inlets) of the chamber 320 of the heat transfer structures 120. As the cold working fluid (WF_{C}) absorbs heat, it becomes a heated working fluid (WF_{H}) at a higher temperature and is directed through an outlet 142 (e.g., one of several outlets) of the chamber 320 of the heat transfer structures 120 to the heated working fluid passage 140. The heated working fluid passage 140 and the cold working fluid passage 130 are each fluidically connected to the heat exchanger 160 and are part of a closed loop system. As such, heated working fluid (WF_{H}) from the heated working fluid passage 140 flows into the heat exchanger 160 at a higher pressure than the cold working fluid (WF_{C}), and the cold working fluid (WF_{C}) is thereby driven from the heat exchanger 160 to the cold working fluid passage 130 through which it flows to each of the heat transfer structures 120 in a continuous cycle. The heat exchanger 160 is shown schematically in FIG. 2. In some embodiments, the heat exchanger 160 can be positioned radially peripheral to each of the cold working fluid passage 130 and heated working fluid passage 140 (e.g., the outermost element of the heat transfer system) and radially inward of the insulation member 150. In some embodiments, the heat exchanger 160 can be positioned physically above the heat transfer structures 120 such the cold working fluid (WF_{C}) provided from the heat exchanger 160 has additional head pressure, which can beneficially provide better circulation of the cold working fluid (WF_{C}) through the heat transfer structures 120.

The individual heat transfer structures 120 of FIG. 2, (and corresponding areas of the cold working fluid passage 130 and heated working fluid passage 140) can have different orientations. For example, some of the heat transfer structures 120 are positioned substantially parallel to gravitation force, other heat transfer structures 120 are positioned at an angle relative to gravitational force, and yet other heat transfer structures 120 are positioned substantially perpendicular to gravitational force. Accordingly, in some embodiments the heat transfer system 115 can operate despite these different orientations and/or be substantially insensitive to gravitational forces acting on the device 100. That is, the heat transfer system 115 and its individual elements (e.g., the heat transfer structures 120) can operate irrespective of their orientation to gravitational force.

The insulation member 150 can be the outermost layer or element and/or it can be peripheral to the heat transfer system, and it can fully or partially enclose the other elements of the device 100. The insulation member 150 can prevent or inhibit heat leakage from the device 100 to the ambient environment and/or from the ambient environment to the device 100. In practice, the insulation member 150 can also serve as a protective barrier between the user (e.g., the human 10) and the other elements of the device 100, which can have more extreme temperatures.

In some embodiments, the insulation member 150 can have additional functionality and/or serve other functions. For example, in some embodiments the insulation member 150 can be configured to contain compressed air (or other fluid) with an adjustable pressure to increase and/or decrease the contact pressure applied from the device 100 on the target area of the human 10. Altering such pressure can alter blood flow to and/or from the target area, which can be beneficial for treating swelling and/or pain. For example, in some embodiments the device 100 can cool the target area of the human 10 for a period of time (e.g., 15-20 minute) at a pressure (e.g., compression) applied via the insulation member 150 or other member of the device 100, and then cease thermal cooling and decrease the applied pressure for a period of time (e.g., 5-10 minutes). By decreasing the applied pressure, blood flow to the target area is enhanced, while the target area is in a cooled state. Additionally or alternatively, the ability to adjust the applied pressure of the device, and therein the compressive force the device is applying to the target area, can eliminate the need to remove and refasten the device 100.

The TEC 110 of the device 100 can include a thermoelectric first face 312 at a first side of the TEC 110 and adjacent the flexible support unit 105, a thermoelectric second face 316 at a second opposing side of the TEC 110 and adjacent the heat transfer structure 120, and thermoelectric legs or pillars 314 extending between the first face 312 and the second face 316. In some embodiments the second face 316 may be omitted and the legs 314 are in direct contact with the heat transfer structure 120. As shown in FIG. 2, the TEC 110 and the heat transfer system 115 including the heat transfer structures 120, cold working fluid passage 130, and heated working fluid passage 140 can together have a dimension (D₃) of no more than 1 mm, 3 mm, 5 mm, 10 mm, 15 mm, 25 mm, or 30 mm, or within a range of 1 millimeter (mm) to 30 mm or any incremental range therebetween. In some embodiments, the TEC 110 and the heat transfer structure 120 together can have a dimension (D₄) of no more than 1 mm, 3 mm, 5 mm, 10 mm, 15 mm, 25 mm, or 30 mm, or within a range of 1 mm to 30 mm or any incremental range therebetween.

In some embodiments, the TECs 110 (e.g., the first face 312, the second face 316, and/or the legs 314) can comprise a rigid material that is generally inflexible. In such embodiments it can be desirable to limit the footprint of individual TECs 110 to maintain the overall flexibility of the device 100 (or any other heat transfer device disclosed herein) and ensure it can conform around or to the geometry of a target area (e.g., the knee). That is, when the footprint of the TECs 110 is smaller, and therein the rigid portions of the device 100 are smaller, the device 100 can have sufficient flexibility between the TECs 110 from the flexible support unit 105 to at least generally conform around or to the geometry of a target area to improve thermal contact between the human and the TECs 110. In some embodiments, the TECs can have a footprint (e.g., surface area over the flexible support unit 105) of no more than 2 mm², 3 mm², 4 mm², 5 mm², 6 mm², 7 mm², 8 mm², or 9 mm², or within a range of 2-9 mm² or any incremental range therebetween.

In some embodiments, the first face 312, the second face 316, and/or the legs 314 of individual TECs 110 can comprise a flexible material, e.g., to enable the TECs 110 to better conform to a target area when the device 100 is worn by a human. Relative to those embodiments in which the TECs 110 are formed of rigid materials, using a flexible material, e.g., for the first face 312 (i.e., the hot side) of the TEC 110 can enable the foot print of the TEC 110s to be larger since the flexibility of the device 100 is no longer limited by the TECs 110. In doing so, the larger heat TECs 110 can enable a higher capacity for heat transfer and/or decrease manufacturing costs for the device 100.

FIG. 3A is a cross-sectional isometric view of a portion of the device 100, in accordance with embodiments of the present technology. Only the TEC 110 and heat transfer structures 120 are shown in FIG. 3A and other elements of the device 100 are omitted for illustrative purposes. As shown in FIG. 3A, the heat transfer structure 120 has microfeatures 324 defined by continuous elongated walls that form continuous elongated channels 326 arranged in multiple rows. The channels 326 can be substantially identical to one another and have a uniform width along its length. In some embodiments, the channels 326 can have widths that vary along their length, e.g., becoming narrower as they approach an inlet or outlet of the chamber. Additionally or alternatively, individual channels may differ (e.g., be wider or narrower) from adjacent channels. Without being bound by theory, such channel design can induce additional favorable pressure gradients on liquid working fluid flow.

In some embodiments, the microfeatures can comprise structures that are not continuous elongated walls but rather other types of protrusion extending into the chamber. For example, as shown in FIG. 3B, which illustrates a cross-sectional view of a portion of a heat transfer structure, the microfeatures 324 are pillars or pins. As shown in FIG. 3B, the microfeatures 324 can be arranged in rows and columns, or other suitable arrangements, that define channels 326 in the spaces between the microfeatures 326. Although the pin-type microfeatures 324 shown in FIG. 3B have a rectilinear cross-section, they can have circular or other cross-sectional shapes (e.g., hexagonal, octagonal, etc.). As also shown in FIGS. 3A and 3B, the working fluid (WF) within the channels 326 defined by the microfeatures 324 flows from the inlet region 132 (FIG. 3A) to the outlet region 142 (FIG. 3A) where it exits the chamber 320.

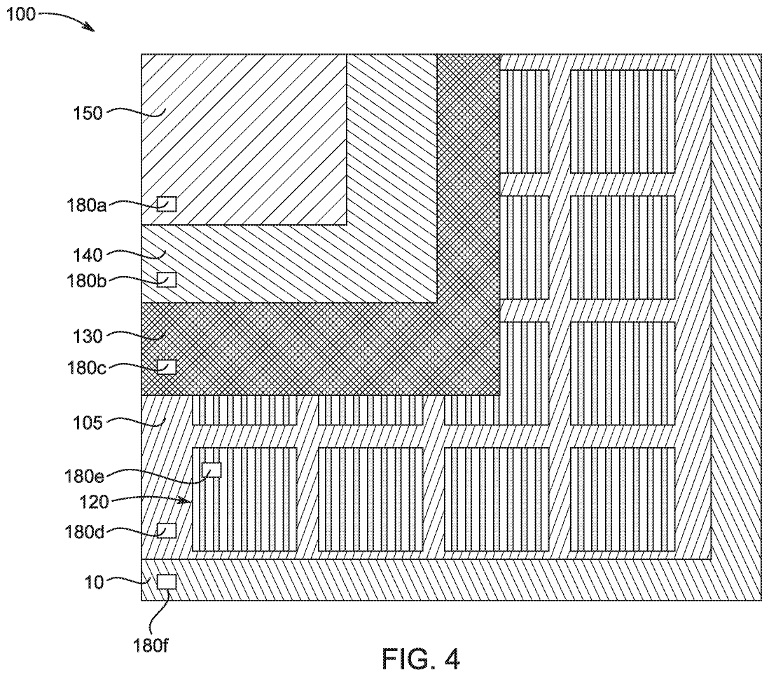

FIG. 4 is a partially schematic top view of the device 100 of FIG. 1, in accordance with embodiments of the present technology. Portions of elements of the device 100 (as shown in FIG. 1) are removed from FIG. 4 to illustrate a layered arrangement of the elements of the device 100. As shown, the device 100 includes, in a radially outward direction, the flexible support unit 105, heat transfer structures 120, the cold working fluid passage 130, the heated working fluid passage 140, and the insulation member 150. For illustrative purposes, the heat exchanger 160 (as shown in FIG. 2) is not shown in FIG. 4 and the TECs 110 are covered by the heat transfer structures 120. The flexible support unit 105 is thermally coupled to and extends between each of the TECs 110. The flexible support unit 105 can comprise a thermally conductive and/or flexible contact member that acts as a heat spreader to enhance heat transfer to and/or from the target area of the human 10 in the regions between the TECs 110. Additionally or alternatively, the flexible support unit 105 can comprise conductive materials and/or biocompatible materials, including metals, metallic alloys, coatings, polymers, silicone, and/or combinations thereof. In some embodiments, the thermally conductive member can comprise a metal sheet or material at a first side of the contact member and in contact with the individual TECs 110, and a non-metal sheet or material at a second opposing side of the thermally conductive member and in contact with the human 10. In some embodiments, the flexible support unit 105 comprises an elastic wrap or material configured to be wrapped around the target area. The elastic wrap can be strapped with a fastener configured to retain the elastic wrap and exert a compressive force against the target area of the human 10. As shown in FIGS. 1 and 2, the TECs 110 are each over the flexible support unit 105, and the flexible support unit 105 is around the human 10. In some embodiments, the flexible support unit 105 extends only between individual ones of the TECs 110 and the TECs 110 are directly over the human 10 (e.g., in direct contact with the human 10). In some embodiments, the flexible support unit 105 can be omitted entirely, and the TEC 110 is over or directly over the human 10.

The device 100 can include one or more sensors 180a-f (collectively referred to as “sensors 180”), which are illustrated schematically in FIG. 4. As shown, the device 100 can include a first sensor 180a on and configured to measure a desired parameter (e.g., temperature, pressure, etc.) of the insulation member 150, a second sensor 180b on and configured to measure a desired parameter of the heated working fluid passage 140, a third sensor 180c on and configured to measure a desired parameter of the cold working fluid passage 130, a fourth sensor 180d on and configured to measure a desired parameter of the flexible support unit 105, a fifth sensor 180e on and configured to measure a desired parameter of the heat transfer structures 120, and a sixth sensor 180f on and configured to measure a desired parameter of the human 10. Other sensors may also be included depending on the end use of the device 100. For example, one or more other sensors can be on and configured to measure a desired parameter of the TECs 110, e.g., to measure individual performance or abnormal operation thereof. Each of the sensors 180 can be in communication with the controller and be used to verify and/or improve safety (e.g., prevent overcooling and/or high pressure zones), efficacy, and operation of the device 100 via the controller.

As shown in the illustrated embodiment of FIG. 4, the device 100 includes eight separate TECs 110. In other embodiments, the actual number of TECs 110 may be more or less (e.g., 2, 3, 5, 10, 20, 30, or more) depending on the particular end use of the device 100 and the heating/cooling capacity requirements needed from the device 100. Further, the shape and size of TECs 110 may vary among different regions of the device. Additionally or alternatively, the TECs 110 may be arranged differently than that shown in FIG. 4. For example, in addition to individual TECs 110 be around a target area (e.g., around a circumference of the human 10) as shown in FIG. 1, individual TECs 110 may be stacked on top of one another to increase the heating and/or cooling ability of that particular stack of TECs 110. In such embodiments, a second TEC 110 stacked on top of a first TEC 110 can have one side in contact with the first TEC 110 and another opposing side in contact with the heat transfer structures 120. The stacked arrangement of TECs 110 can be particularly beneficial when more extreme temperatures (e.g., less than 0° C., −10° C., or −20° C.) at the target area of the human 10 are desired.

FIG. 5A is a partially schematic top view of a single-phase heat transfer system 515 of a wearable heat transfer device (e.g., the device 100; FIG. 1A), and FIG. 5B is a partially schematic cross-sectional side view of the heat transfer system 515 of FIG. 5A. Similar to the heat transfer systems or portions thereof previously described (e.g., with reference to FIGS. 3A-3B), the heat transfer system 515 is over the TECs 110 and configured to remove heat therefrom. Referring to FIGS. 5A and 5B together, the heat transfer system 515 can include a base member or substrate 522 over one or more TECs 110. The substrate 522 can include microfeatures 524 (e.g., pins or other structures configured to increase and exposed surface area of the substrate) that at least partially define channels 526 of a fluid distribution network or manifold 525. The microfeatures 524 and channels 526 can include the features and/or functionality of the respective microfeatures 322 and channels 324 described herein. The channels 524 are configured to receive a cold working fluid (WF_{C}) to absorb heat from the substrate 522 and/or microfeatures 522. The cold working fluid (WF_{C}) can be provided to the individual fluid distribution networks 525 at a first temperature and an inlet 528 positioned at an intermediate or central region thereof, and exit the fluid distribution network as heated working fluid (WF_{H}) at a second temperature higher than the first temperature at outlets 530a-b (collectively referred to as “the outlets 530”) at peripheral regions on opposing sides of the fluid distribution network 525. By providing the working fluid at an intermediate region, the fluid distribution network 525 can provide more uniform cooling relative to a fluid distribution network that supplied the working fluid on a first side and removed heated working fluid from a second opposing side. As shown in FIGS. 5A and 5B, the fluid distribution network 525 includes only one inlet and one outlet 530a, 530b on each side of the fluid distribution network 525. In other embodiments, the fluid distribution network 525 can include multiple inlets, multiple outlets, or multiple inlets and outlets.

In alternative embodiments, the heat transfer system 515 may function in an opposite arrangement where the channels 524 are configured to receive a heated working fluid (WF_{H}) to provide heat the substrate 522 and/or microfeatures 522. The heated working fluid (WF_{H}) can be provided to the individual fluid distribution networks 525 at a first temperature and an inlet 528 positioned at an intermediate or central region thereof, and exit the fluid distribution network as cooled working fluid (WF_{C}) at a second temperature lower than the first temperature at the outlets 530.

The heat transfer system 515 can further include (i) a heat exchanger 560 that cools the heated working fluid (WF_{H}), e.g., to the first temperature, and (ii) one or more pumps 565 configured to circulate the working fluid throughout the heat transfer system 515. The heat exchanger 560 can include features and functionality identical to the heat exchanger 160 described herein.

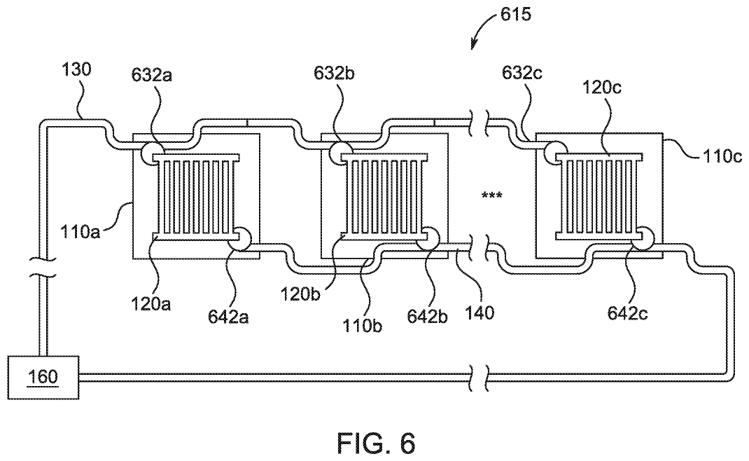

FIG. 6 is a partially schematic top view of a heat transfer system 615 of a heat transfer device (e.g., the heat transfer device 100; FIGS. 1-4), in accordance with embodiments of the present technology. The heat transfer system 615 can correspond to the heat transfer system 115 and include some, similar, or all of the elements previously described in connection with the heat transfer system 115. As shown in FIG. 6, the heat transfer system 615 can include the cold working fluid passage 130 fluidically coupled at an upstream end to the heat exchanger 160 and fluidically coupled at a downstream end to an inlet portion of each of the heat transfer structures 120 (individually shown as heat transfer structures 120a, 120b, and 120c), and can include the heated working fluid passage 140 fluidically coupled at an upstream end to an outlet portion of each of the heat transfer structures 120 and fluidically coupled at a downstream end to the heat exchanger 160. The cold working fluid passage 130 is configured to provide the cold working fluid (WF_{C}) to the heat transfer structures 120 and the heated working fluid passage 140 is configured to receive the heated working fluid (WF_{H}) from the heat transfer structures 120. As previously described, each of the heat transfer structures 120 is thermally coupled to and configured to remove heat from a corresponding one of the TECs 110 (individually shown as TECs 110a, 110b, and 110c).

As also shown in FIG. 6, the cold working fluid passage 130 is at least fluidically coupled to the first heat transfer structure 120a at a first inlet region 632a and the second heat transfer structure 120b at a second inlet region 632b downstream of the first inlet region 632a. The cold working fluid passage 130 may further be fluidically coupled to the third heat transfer structure 120c at a third inlet region 632c downstream of the second inlet region 632b. The heated working fluid passage 140 is at least fluidically coupled to the first heat transfer structure 120a at a first outlet region 642a and the second heat transfer structure 120b at a second outlet region 642b downstream of the first outlet region 642a. The heated working fluid passage 140 may further be fluidically coupled to the third heat transfer structure 120c at a third outlet region 642c downstream of the second outlet region 642b. The heat transfer system 615 may include additional heat transfer structures 120 with associated inlet and outlet regions 632, 642 fluidically coupled to the cold working fluid passage 130 and the heated working fluid passage 140, respectively, downstream of the third heat transfer structure 120c. The cold working fluid passage 130 extends from the heat exchanger 160 to the third (or most downstream) inlet region 632c of the third (or most downstream) heat transfer structure 120c, and the heated working fluid passage 140 extends from the first outlet region 642a of the first heat transfer structure 120a to the heat exchanger 160.

In operation, the cold working fluid passage 130 provides cold working fluid (WF_{C}) from the heat exchanger 160 to all heat transfer structures 120 fluidically coupled to the cold working fluid passage 130 (e.g., heat transfer structures 120a-c, as illustrated), and the heated working fluid passage 140 returns heated working fluid (WF_{H}) to the heat exchanger 160 from all heat transfer structures 120 fluidically coupled to the heated working fluid passage 140 (e.g., heat transfer structures 120a-c, as illustrated). As shown in FIG. 6, the heat transfer system 615 operates as a hybrid series-parallel system. The cold working fluid passage 130 receives cold working fluid (WF_{C}) from the heat exchanger 160 and delivers the cold working fluid (WF_{C}) to (i) the first inlet region 632a at a first inlet temperature and pressure, (ii) the second inlet region 632b at a second inlet temperature and pressure, and (iii) the third inlet region 632c at a third inlet temperature and pressure. The first, second, and third inlet temperatures of cold working fluid (WF_{C}) are the same or substantially the same (e.g., within 1, 5%, or 10%), and the first inlet pressure is greater than the second inlet pressure and the second inlet pressure is greater than the third inlet pressure.

The heated working fluid passage 140 receives heated working fluid (WF_{H}) from (i) the first outlet region 642a at a first outlet temperature and pressure, (ii) the second outlet region 642b at a second outlet temperature and pressure, (iii) and the third outlet region 642c at a third outlet temperature and pressure. The first outlet pressure is greater than the second outlet pressure and the second outlet pressure is greater than the third outlet pressure. Additionally, the pressure difference between the first inlet and outlet regions 632a, 642a is the same or substantially the same as the pressure difference between the second inlet and outlet regions 632b, 642b and as the pressure difference between the third inlet and outlet regions 632c, 642c. This equal pressure difference causes an equal or substantially equal flowrate of working fluid to pass through the first, second, and third heat transfer structures 120a-c. If additional heat transfer structures 120 are included in the heat transfer system 615, (i) their inlet regions also (a) receive cold working fluid (WF_{C}) from the heat exchanger 160 at the same temperature as the prior inlet regions 632a-c and (b) have a lower inlet pressure than the prior inlet regions 632a-c, and (ii) their outlet regions also (a) have a lower outlet pressure than the prior outlet regions 634a-c and (b) have the same pressure difference between inlet and outlet pressures as the heat transfer structures 120a-c. In some embodiments, the pressure difference between inlet and outlet pressures for individual heat transfer structures 120a-c can be less than 1.0 bar or 0.5 bar, or within a range of 0-1 bar or 0-0.5 bar.

Embodiments of the present technology described with reference to FIG. 6 have multiple advantages over other conventional heat transfer devices that use cooling systems connected in parallel or series to heat transfer structures. For example, the closed loop system illustrated and described enables embodiments of the present technology to provide thermal treatment by cooling at an even temperature across the target area and more efficient system operation overall. As described herein, this is achieved because the heat transfer structures are arranged relative to the cold working fluid passage 130 and hot working fluid passage 140 such that the working fluid provided to each heat transfer structure has the same or substantially the same temperature, and the pressure drop across the heat transfer structures is the same or substantially the same. In doing so, flow rate of the working fluid across the individual heat transfer structures is the same or similar to one another. As a result, the TECs of the heat transfer device, regardless of location relative to an incoming supply header, can be effectively thermally treated.

In contrast the embodiment shown in FIG. 6, for heat transfer devices utilizing a series arrangement, downstream heat transfer structures of the heat transfer system receive a working fluid that is at a higher temperature due to heat absorbed from upstream heat transfer structures. That is, as working fluid is passed consecutively through the heat transfer structures, each downstream heat transfer structure receives warmer working fluid than the prior structure. As a result, the downstream heat transfer structures are less effective than the upstream heat transfer structures at removing heat from the corresponding TECs. For heat transfer devices utilizing a parallel arrangement, the pressure drop across the heat transfer structures experienced by the working fluid is equal only if the working fluid supply header to the individual heat transfer structures is centrally located between the heat transfer structures. If for example, the working fluid supply header is closer to one heat transfer structure than another, the working fluid will prefer the path of least resistance, and the further heat transfer structure will receive less working fluid. Additionally, less heat will be absorbed from the corresponding TEC. For heat transfer devices configured to be worn by humans and thus wrapped around a body part or target area, the ability to centrally locate a supply header can be difficult and, in some instances, not practical.

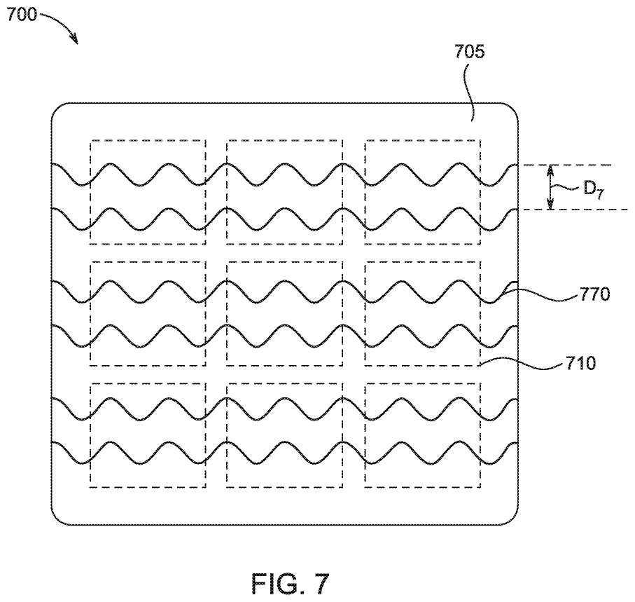

FIG. 7 is a partially schematic bottom view of a heat transfer device 700 (“device 700”), in accordance with embodiments of the present technology. The heat transfer device 700 can correspond to the device 100 and include some, similar, or all of the elements previously described in connection with device 100. The device 700 can include a flexible support unit 705, modules 710 (shown via dashed lines) over a top surface of the flexible support unit 705, and thermally conductive members 770. The modules 710 can include the TECs 110 and/or heat transfer structures 120 previously described. The thermally conductive members 770 can be embedded within (e.g., woven or sown into) or coupled to the flexible support unit 705, and can extend laterally across all or part of a dimension of the flexible support unit 705. In some embodiments, as shown in FIG. 7, the thermally conductive members 770 can extend over and/or be generally aligned with a row of modules 710. In some embodiments, the thermally conductive members 770 can extend between certain rows or groups of modules 710 or between certain individual modules 710. Adjacent thermally conductive members 770 can be spaced apart from one another by a dimension (D₇), which can be at least 0 mm, 2 mm, 4 mm, 6, mm, 8 mm, or 10 mm, or within a range of 0-10 mm, or any incremental range therebetween. As shown in FIG. 7, two thermally conductive members 770 are included adjacent one another for each row of the modules 710. In some embodiments, more (e.g., three, five, etc.) or fewer thermally conductive members 770 may be included. The thermally conductive members 770 can comprise or consist of a metal material (e.g., copper, brass, steel, zinc, or alloys thereof) or other synthetic materials that are conductive and have a higher rigidity than that of the flexible support unit 705.

As shown in FIG. 7, the thermally conductive members 770 can have a serpentine shape (e.g., wave-like, oscillating, etc.) as they extend across the device 700. The wire can be wire laid or metal formed into the serpentine shape, stamped into the serpentine shape, or manufactured using any other suitable manufacturing process that may achieve a desired pattern before the thermally conductive members 770 are incorporated with the device 700. In some embodiments, the thermally conductive members 770 can be adhered to, in the radial direction, a bottom or top surface of the flexible support unit 705. In all described implementations of thermally conductive members 770, the thermally conductive members 770 distribute heat across the flexible support unit 705, modules 710, or groups of modules 710, such that the thermal effects from the modules 710 (e.g., the TECs) are more uniform across a dimension of the flexible support unit 705. Additionally or alternatively, the thermally conductive members 770 may improve strength and elasticity of the device 700 and/or flexible support unit 705.

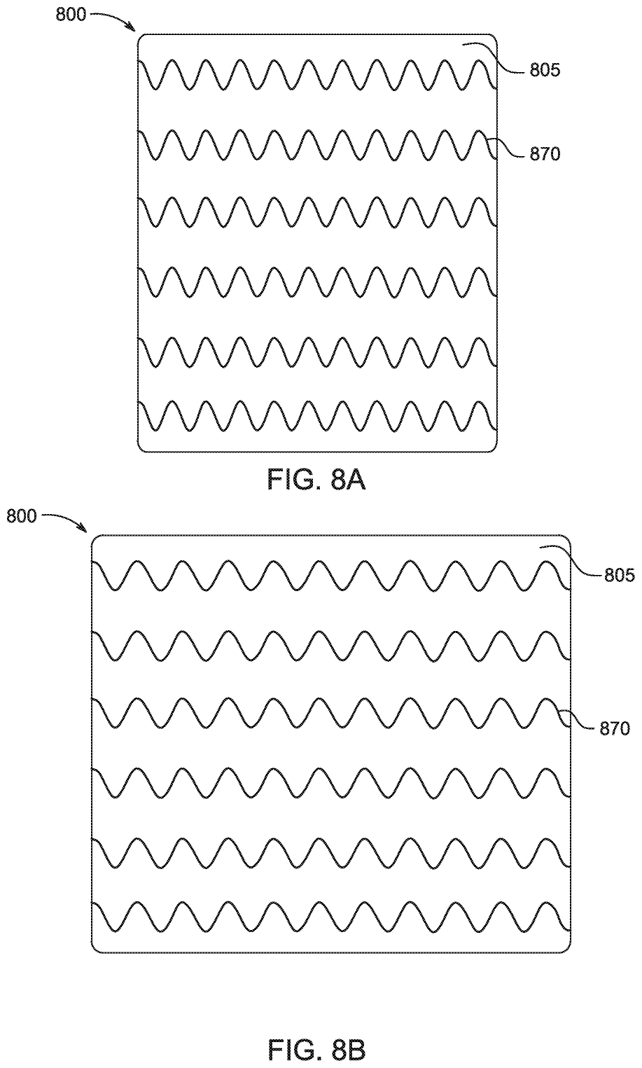

Improvements to strength and elasticity provided by thermally conductive members are further illustrated in FIGS. 8A and 8B, which are partially schematic bottom views of a heat transfer device 800 (“device 800”). The heat transfer device 800 can correspond to the device 100 and include some, similar, or all of the elements previously described in connection with device 100. Referring to FIGS. 8A and 8B, the device 800 includes a flexible support unit 805 that is expandable (e.g., elastic, stretchable, etc.) along one or more dimensions, and includes thermally conductive members 870 that extend along all or part of a dimension of the flexible support unit 805. The thermally conductive members 870 can correspond to the thermally conductive members 770 and include some, similar, or all of the elements previously described in connection with the thermally conductive members 770. In FIG. 8A the flexible support unit 805 is in an unexpanded state (e.g., relaxed state), and in FIG. 8B the flexible support unit 805 is in an expanded state (e.g., tensioned state).

The thermally conductive members 870 can provide strength to the flexible support unit 805, or more generally to the device 800, by introducing a material with higher rigidity than the flexible support unit 805 into the flexible support unit 805. In the unexpanded state of the device 800 shown in FIG. 8A, the thermally conductive members 870 are relaxed, distributing heat and providing structure to the flexible support unit 805 and the device 800. Additionally or alternatively, the thermally conductive members 870 can provide elasticity by acting as a spring within the flexible support unit 805. When the thermally conductive members 870 and the flexible support unit 805 are expanded along a dimension of the thermally conductive members 870, the device 800 enters the expanded state shown in FIG. 8B (as illustrated by the increased wavelength of the thermally conductive members 870) and the thermally conductive members 870 generate a spring force to bias the device 800 toward the unexpanded state, in addition to providing continued heat distribution and structure to the flexible support unit 805.

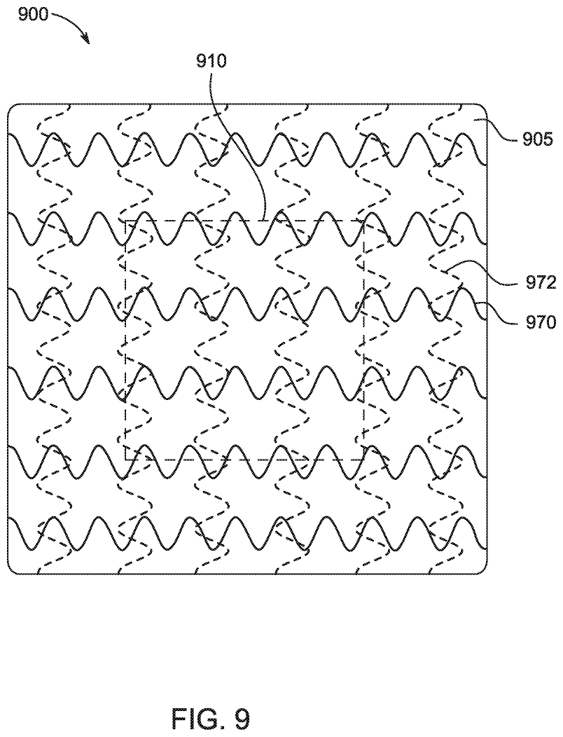

FIG. 9 illustrates an alternative approach to providing improvements to heat dissipation, strength and elasticity as previously illustrated in FIGS. 7-8B. FIG. 9 is a partially schematic bottom view of a heat transfer device 900 (“device 900”) in accordance with embodiments of the present technology. The heat transfer device 900 can correspond to the device 100 and include some, similar, or all of the elements previously described in connection with device 100. The device 900 can include a flexible support unit 905, modules 910 (a single module is shown using dashed lines) over a top surface of the flexible support unit 905, first thermally conductive members 970, and second thermally conductive members 972. The modules 910 can include the TECs 110 and/or heat transfer structures 120 previously described. The flexible support unit 905 can be expandable (e.g., stretchable) along at least a first and second dimension and include the first and second thermally conductive member 970, 972 that may respectively extend along at least the first and second dimension of the flexible support unit 905.

The first and second thermally conductive members 970, 972 both or individually can correspond to the thermally conductive members 770. In particular, the first and second thermally conductive members 970, 972 may similarly be (i) embedded within or coupled to the flexible support unit 905, (ii) extend across all or part of a dimension of the flexible support unit 905, (iii) extend over and/or be generally aligned with a row of modules 910, and/or (iv) extend between certain individual, rows, or groups of modules 910. The first and second thermally conductive members 970, 972 both or individually may similarly comprise or consist of a metal material (e.g., copper, brass, steel, zinc, or alloys thereof) or other synthetic materials that are conductive and have a higher rigidity than that of the flexible support unit 905. The first and second thermally conductive members 970, 972 both or individually may similarly have a wave-like or oscillating shape as they extend across the device 900 and be laid, metal formed, or stamped into the waved shape. The first and second thermally conductive members 970, 972 both or individually may similarly be adhered to, in the radial direction, the bottom or top surface of the flexible support unit 905. The first and second thermally conductive members 970, 972 both or individually may similarly distribute heat across the flexible support unit 905, module 910, or groups of modules 910, such that the thermal effects from the modules 910 (e.g., the TECs) are more uniform across one or more dimension of the device 900.

As shown in FIG. 9, four respective first and second thermally conductive members 970, 972 extend across the module 910. In some embodiment, more (e.g., five, six, etc.) or fewer first and/or second thermally conductive members 970, 972 may be included. The first and second thermally conductive members 970, 972 can be positioned within the flexible support unit 905 and intersect substantially at right angles to one another. In some embodiments, the first and second thermally conductive members 970, 972 may intersect at substantially acute angles. In some embodiments, the device 900 may include additional thermally conductive members intersecting with the first and the second thermally conductive members 970, 972. In these further embodiments, the included thermally conductive members can intersect with all other included thermally conductive members at substantially 60° angles, at substantially 90° angles with some and substantially 45° with other included thermally conductive members, or at any other combination of intersection angles.

The first and second thermally conductive members 970, 972 may improve strength of the flexible support unit 905, or more generally the device 900, by introducing materials with higher rigidity than the flexible support unit 905 into the flexible support unit 905. Additionally or alternatively, the first and second thermally conductive members 970, 972 may improve elasticity of the device 900 along all or part of at least the first and second dimensions of the flexible support unit 905. For example, as similarly illustrated in FIG. 8, when the first thermally conductive members 970 and the flexible support unit 905 are expanded, the first thermally conductive members 970 can bias the device 900 to return to an unexpanded state from an expanded state along the first dimension of the flexible support unit 905 corresponding with the first thermally conductive members 970. Likewise, when the second thermally conductive members 972 and the flexible support unit 905 are expanded, the second thermally conductive members 972 can bias the device 900 to return to an unexpanded state from an expanded state along the second dimension of the flexible support unit 905 corresponding with the second thermally conductive members 972.

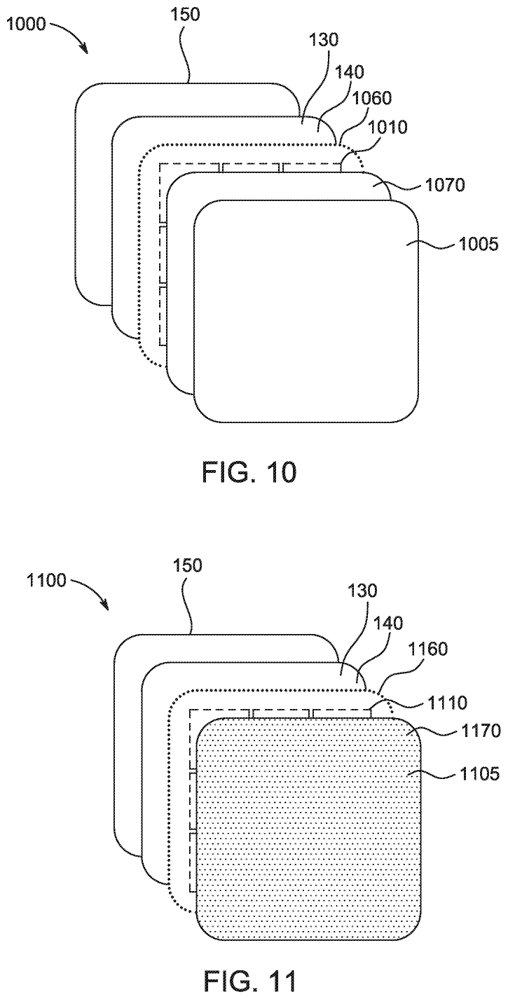

In some embodiments, the thermally conductive members 770, 870, 970 may be a continuous structure of highly conductive material (e.g., a sheet) or highly conductive particles dispersed within and/or on the flexible support unit (e.g., the flexible support unit 105, 705, 805, 905). FIG. 10 is a schematic exploded isometric view of a heat transfer device 1000 (“device 1000”) with a thermally conductive member 1070 illustrated as a sheet of highly conductive material, in accordance with embodiments of the present technology. The heat transfer device 1000 can correspond to the device 100 and include some, similar, or all of the elements previously described in connection with device 100. In FIG. 10, the layers of the device 1000 are exposed, in-part, to show the flexible support unit 1005, the thermally conductive member 1070, modules 1010 (shown via dashed lines), a group of modules 1060, the cold working fluid passage 130, the heated working fluid passage 140, and the insulation member 150. The modules 1010 can include the TECs 110 and/or heat transfer structures 120 previously described. As shown in FIG. 10, the thermally conductive member 1070 is a sheet of highly conductive material extending across multiple modules 1010 of the module group 1060. The thermally conductive member 1070 is adhered, in the radial direction, to the top of the flexible support unit 1005 or alternatively adhered to the modules 1010 before combination with the flexible support unit 1005. The thermally conductive member 1070 may alternatively be adhered, in the radial direction, to the bottom of the flexible support unit 1005. The thermally conductive member 1070 may be a uniform sheet extending in all directions between the modules 1010 and the flexible support unit 1005. Additionally or alternatively, areas of the thermally conductive member 1070 (e.g., areas overlapping modules 1010) can vary in thickness or have cutouts to avoid or reduce thermal conductivity between certain modules 1010 or for other functional purposes.

FIG. 11 is a schematic exploded isometric view of a heat transfer device 1100 (“device 1100”) with thermally conductive members 1170, in accordance with embodiments of the present technology. The heat transfer device 1100 can correspond to the device 100 and include some, similar, or all of the elements previously described in connection with device 100. In FIG. 11, the layers of the device 1100 are exposed, in-part, to show the flexible support unit 1105, the thermally conductive members 1170, modules 1110 (shown via dashed lines), a group of modules 1160, the cold working fluid passage 130, the heated working fluid passage 140, and the insulation member 150. The modules 1110 can include the TECs 110 and/or heat transfer structures 120 previously described. As shown in FIG. 11, the thermally conductive members 1170 are highly conductive particles interspersed within the flexible support unit 1105. In some embodiments, the thermally conductive members 1170 may be sprayed or otherwise adhered, in the radial direction, to a top or bottom surface of the flexible support unit 1105. The thermally conductive members 1170 can comprise or consist of a metal material (e.g., copper, brass, steel, zinc, or alloys thereof) or other synthetic materials that are conductive and can have a higher rigidity than that of the flexible support unit 1105. In either the illustration of FIG. 10 or 11, the thermally conductive members 1070, 1170 can distribute heat across a dimension of the flexible support unit 1005, 1105, or between modules 1010, 1110 or groups of modules 1060, 1160; and improve strength and/or elasticity of the flexible support unit 1005, 1105.

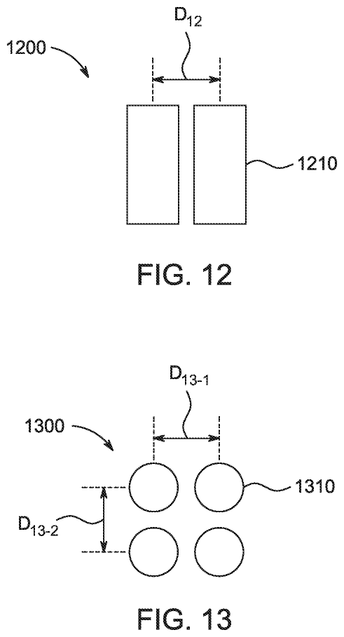

FIGS. 12-14 show alternative embodiments of the arrangement, number, and shape of modules (e.g., modules 110, 710, 810, 910, 1010, 1110). For illustrative purposes of the following description, only outlines of modules are shown in FIGS. 12-14. The modules shown and described with references to FIGS. 12-14 can enable more effective heat treatment of the corresponding devices. For example, the shape of an individual module or arrangement of a group of modules can be determined based on the target area of the human to be treated. For instance, an individual module or group of modules may be utilized such that the shape of that individual module or group of modules is similar to a shape of the target area.

FIG. 12 is a schematic top view of an arrangement of grouped rectangular modules 1210 within a heat transfer device 1200, in accordance with embodiments of the present technology. The heat transfer device 1200 can correspond to the device 100 and include some, similar, or all of the elements previously described in connection with device 100. The modules 1210 can include the TECs 110 and/or heat transfer structures 120 previously described. As shown in FIG. 12, the two modules 1210 are parallel rectangles and are spaced from one another in a first direction (D₁₂). The modules 1210 can be (i) equal in height and width, (ii) equal only in height or width, or (iii) vary in height and width. The modules 1210 may be spaced and centrally aligned, as shown in FIG. 12, or the modules 1210 may be spaced and misaligned, creating a stepped or cascading arrangement. Further, the modules 1210 may not be arranged in parallel, instead the modules 1210 may be arranged askew, e.g.,a with an acute angle therebetween.

FIG. 13 provides an additional illustration of a module arrangement (e.g., module 110, 710, 810, 910, 1010, 1110, 1210). FIG. 13 is a schematic top view of an arrangement of grouped circular modules 1310 within a heat transfer device 1300, in accordance with embodiments of the present technology. The heat transfer device 1300 can correspond to the device 100 and include some, similar, or all of the elements previously described in connection with device 100. The modules 1310 can include the TECs 110 and/or heat transfer structures 120 previously described. As shown in FIG. 13, the modules 1310 of heat transfer device 1300 are circular in shape and roughly arranged in a square configuration, with individual modules 130 spaced apart from neighboring modules 130 by a first distance (D₁₃₋₁) and a second distance (D₁₃₋₂). Fewer modules 1310 may be arranged roughly in a row, column, or other configuration (e.g., a triangle), or more modules 1310 may be arranged according to other polygons. The modules 1310 may be equal in diameter or may vary in diameter. The modules 1210, 1310 of FIGS. 12 and 13, in some embodiments, may have shapes other than a square or circle including, for example, polygons with three or more sides, ovals, or other customized shapes for a specific heat transfer device 1200, 1300 application.

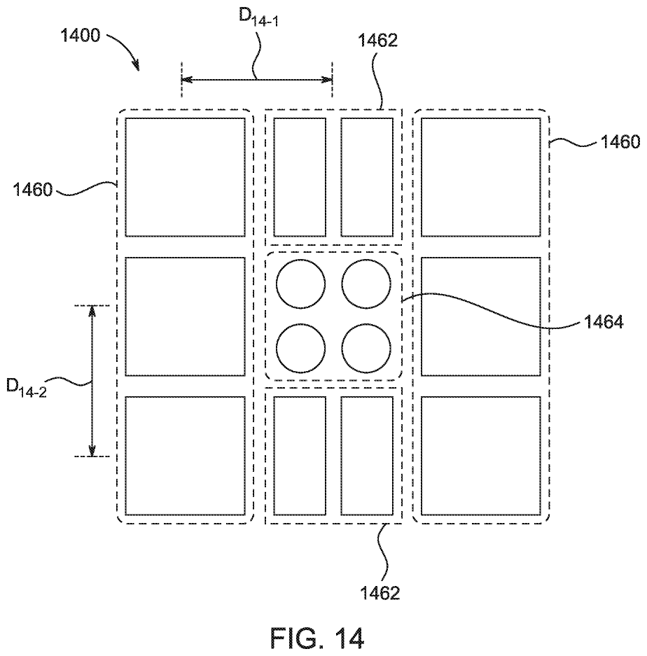

Groups of different modules having different shapes, sizes, spacing and arrangements (e.g., module 110, 710, 810, 910, 1010, 1110, 1210, 1310) may be combined for different heat transfer device applications. FIG. 14 is a schematic top view of arrangements of module groups 1460, 1462, 1464 within a heat transfer device 1400 (“device 1400”), in accordance with embodiments of the present technology. The heat transfer device 1400 can correspond to the device 100 and include some, similar, or all of the elements previously described in connection with device 100. The illustrated device 1400 has square modules groups 1460, rectangular modules groups 1462, and a circular modules group 1464 (collectively, the “groups 1460-1464,” identified by dashed lines). The modules within in the groups 1460-1464 can include the TECs 110 and/or heat transfer structures 120 previously described. The groups 1460-1464 can be arranged within three evenly spaced (D₁₄₋₁) columns. The square groups 1460 can be located in a first and a third column, laterally bookending the device 1400. The rectangular groups 1462 can be evenly spaced (_{D14-2}) above and below the circular group 1464 in a second column. The device 1400 may include additional, individual groups 1460-1464 above, below, or to the side of the device 1400, similarly oriented to the relevant individual group 1460-1464 of FIG. 14 (e.g., vertical rectangles side-by-side). In some embodiments, the additional individual groups 1460-1464 may be rotated from the orientation of FIG. 14 (e.g., horizontal rectangles stacked one above the other). The device 1400 may instead include additional sets of the groups 1460-1464 above, below, or to the side of the device 1400, as oriented in FIG. 14 or rotated therefrom. Each group 1460-1464 may individually implement the heat transfer system 615 of FIG. 6 to connect the TECs of each group 1460-1464. In this structure, each group 1460-1464 may be individually controlled to offer a different heating or cooling therapy, or rate thereof, to the proximate target area of a human 10.

As previously discussed, the modules (e.g., modules 110, 710, 810, 910, 1010, 1110, 1210, 1310, or modules within the groups 1460-1464) described herein can cycle between heating and cooling modes. When the modules described herein are implemented in the device 1400 with different shapes and arrangements, and/or when the device 1400 includes a flexible support unit similar to the flexible support unit 105, the device 1400 can at least substantially conform to the contour of the human 10 (FIGS. 1 and 2). When the device 1400 is in better contact with the human 10, heat transfer from the human 10 to and from the device 1400 is more effective and may more effectively perform thermal therapy to the target area on the human 10 to treat conditions such as pain, swelling, overheating, diminished blood perfusion, diminished nerve connectivity, and/or stroke, while mitigating damage to the epidermal and/or dermal tissues.

### III. Wearable Heat Transfer Device Areas of Treatment

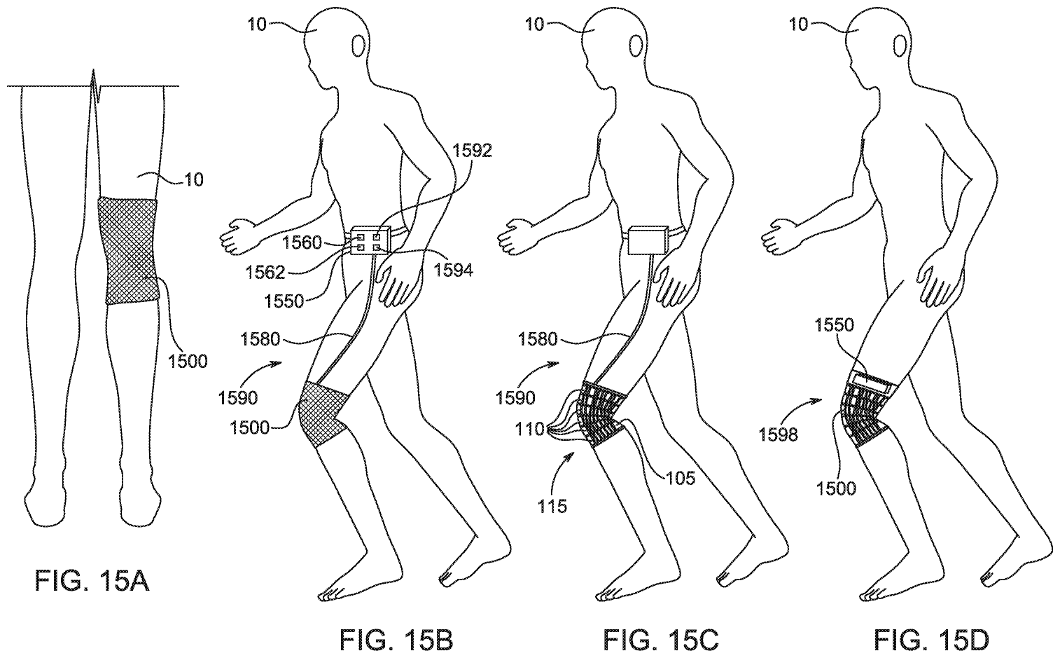

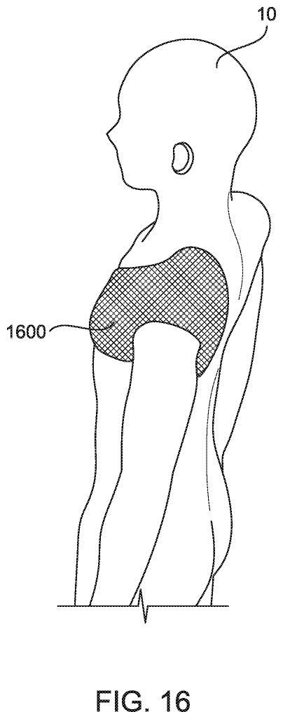

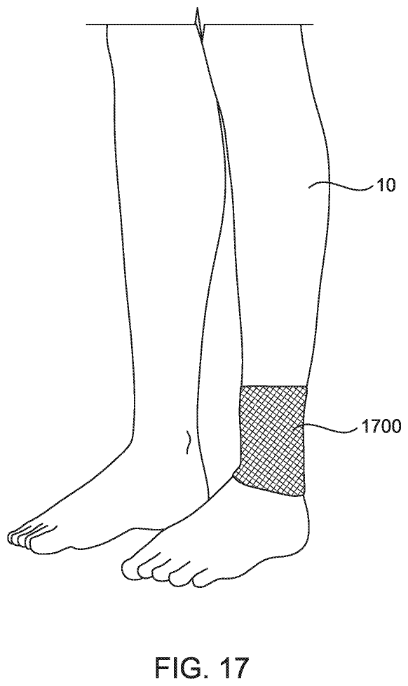

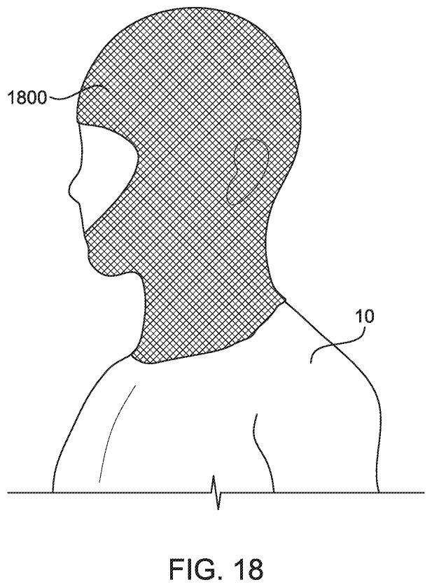

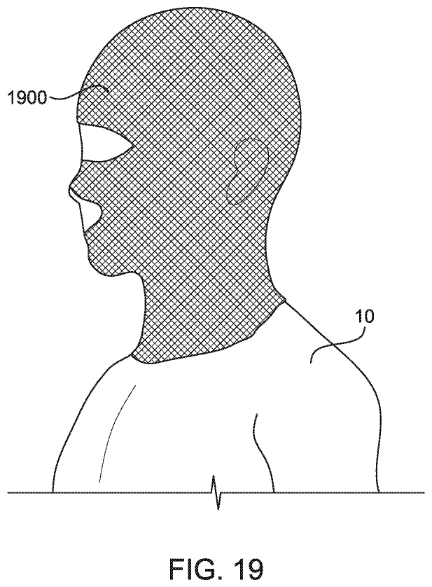

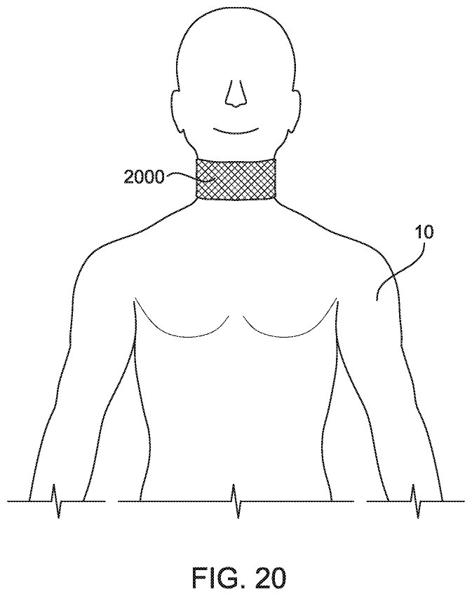

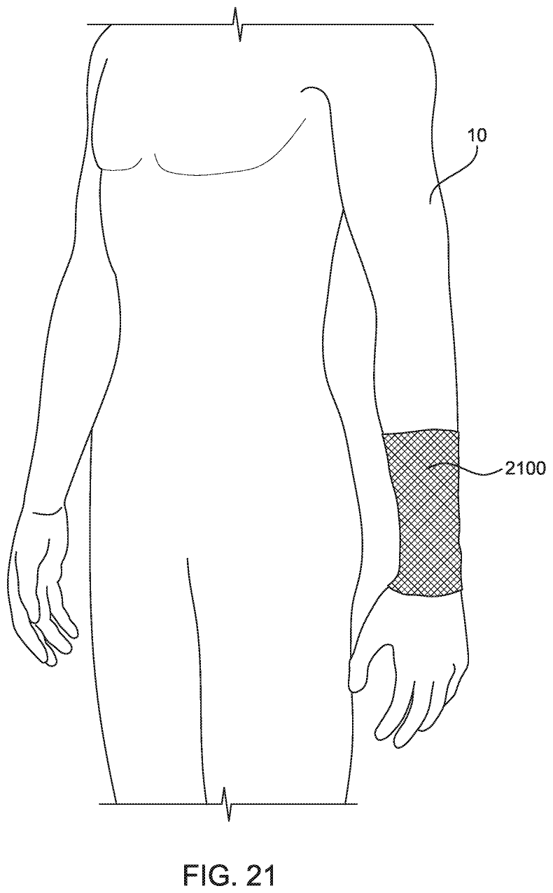

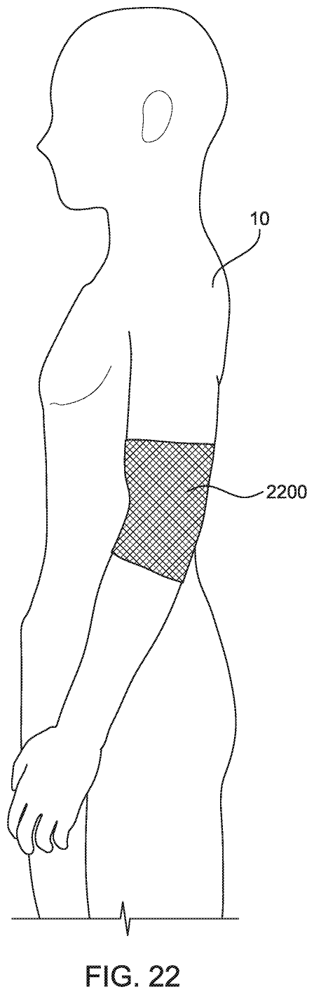

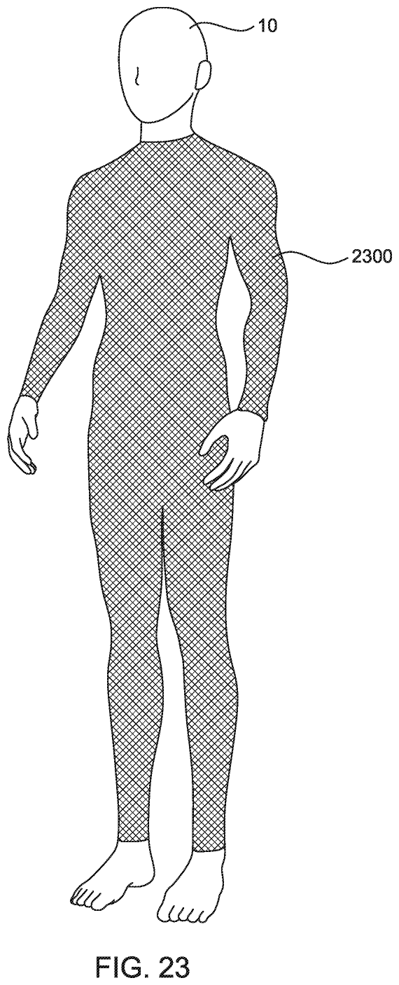

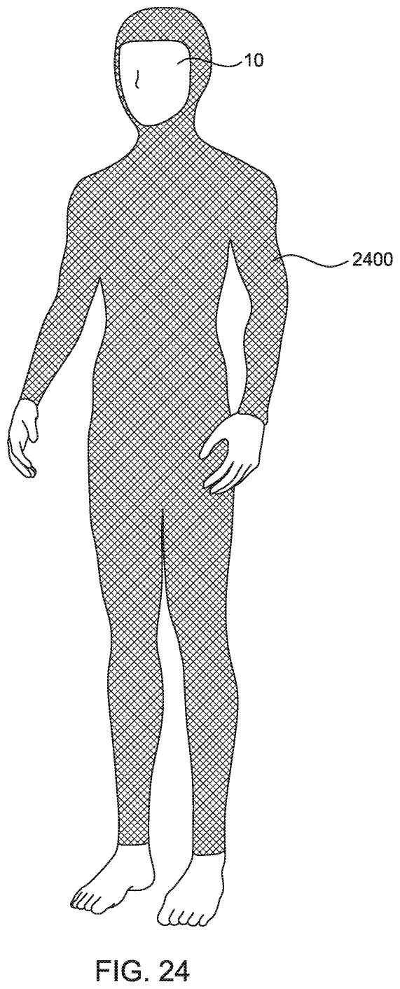

The wearable heat transfer devices disclosed herein can be designed for different target areas and/or body parts, including the head, neck, chest, shoulder, upper back, lower back, upper arm, lower arm, wrist, waist, upper leg, lower leg, feet, hands, etc. The devices can be placed on the target area utilizing fasteners, adhesives, straps, tape (e.g., Velcro), belts, or other means. Some of the target areas are illustrated in FIGS. 15A-24, which are various partially schematic views of the heat transfer devices being worn by a human 10. The devices shown in FIGS. 15A-24 can correspond to any of the devices 100 described herein, and thus can each include some or all of the elements (e.g., the flexible support unit, TECs, cold working fluid passage, heated working fluid passage, etc.) described herein. With reference to these figures, the device 1500 is around a knee region of the human 10, the device 1600 is over a shoulder region of the human 10, the device 1700 is around an ankle and/or lower leg region of the human 10, the device 1800 is around head and neck regions of the human 10, the device 1900 is around head, neck, and facial (e.g., nasal) regions of the human 10, the device 2000 is around a neck region of the human 10, the device 2100 is around a wrist and/or lower arm region of the human 10, the device 2200 is around an elbow region of the human 10, the device 2300 is around lower and upper body regions of the human 10, and the device 2400 is around lower body, upper body, and head regions of the human 10.

FIGS. 15B-15D are partially schematic views of a heat transfer system 1790 including the heat transfer device 1500 and subsystems or other device elements. In addition to the device 1500, the systems described with reference to FIGS. 15B-15D can apply to or be incorporated with any of the devices (e.g., the device 100, 700, 800, 800, 900, 1000, 1100, 1200, 1300 1400, 1500, 1600, 1700, 1800, 1900, 2000, 2100, 2200, 2300, 2400) disclosed herein. The subsystems and/or device elements can be integrated into a package 1550 secured to the human 10 (e.g., at a waist region). The device 1500 can include a heat exchanger 1560 and one or more pumps 1562, which can both be stored in the package 1550. The heat exchanger 1560 can include a liquid-air heat exchanger (e.g., the heat exchanger 560). Storing the heat exchanger 1560 physically above the device 1500 can advantageously provide additional head pressure to the working fluid supplied to the device 1500 and help ensure adequate flow of the working fluid to the fluid distribution network of the device 1500. The one or more pumps 1562 can be fluidically coupled to the heat exchanger 1560 and the heat transfer system of the device 1500, and can ensure adequate flow of the working fluid throughout the heat transfer system.

The heat transfer system 1590 can further include a power source 1592 operably coupled to the device 1500 and configured to provide power to the TECs 110 (FIG. 15C). The power source 1592 can enable the TECs 110 to be set to a particular temperature for heating and/or cooling purposes. In some embodiments, the power source 1592 can include a portable energy storage device (e.g., a battery).

The heat transfer system 1590 can further include a controller and/or electronic component(s) 1594 operably coupled to the device 1500, power source 1592, and other subsystems. In some embodiments, the controller and/or electronic component(s) 1594 can include a transmitter and/or receiver enabling the controller 1594 to communicate (e.g., wirelessly communicate) with a remote user interface (e.g., on a mobile device and/or remote network) and/or the device 1500. In some embodiments, the controller 1594 can be configured to operate the device 1500 in multiple operating modes (e.g., a cooling mode, a heating mode, or both), and/or provide a process value (e.g., a set temperature) at which the device 1500 is configured to operate. In some embodiments, the controller 1594 can provide a setpoint temperature within a range of 40° C. to −20° C. (e.g., 35° C., 20° C., 0° C., −10° C., etc.) to the device 1500 such that the TECs (e.g., the first or second side of the TECs) are configured to operate at the setpoint temperature. Additionally or alternatively, the controller 1594 can be configured to receive inputs from sensors (e.g., sensors 180a-f; FIG. 2) on the device 1500 and control the device based on the received inputs. For example, the controller 1594 can determine any abnormalities of the operating device and automatically generate indications of the abnormalities and/or adjust the operating parameters of the device. Additionally or alternatively, the controller 1594 may utilize artificial intelligence and/or machine learning to adjust power and/or other control parameters, e.g., based on previous treatments used for the same user or a group of users.

In some embodiments, the heat transfer system 1590 can include a conduit 1580 extending from the package 1550 to the device 500. The conduit 1580 can include (i) fluid transport lines, e.g., extending from and fluidically coupling the heat exchanger 1560 and/or one or more pumps 1562 to the fluid distribution network of the device 1500, (ii) power lines, e.g., extending from and operably coupling the power source 1592 to the TECs, and/or (iii) other wires, e.g., extending from and operably coupling the controller to sensors on the device 1500. In some embodiments, the conduit 1580 is omitted, e.g., as shown and described with reference to FIG. 10D. Additionally or alternatively, in some embodiments in which the conduit 1580.

FIG. 15C illustrates another view of the system 1590 shown and described with reference to FIG. 15B, but omits an outer cover of the device 1500 for illustrative purposes. As such, the TECs 110, flexible support unit 105, and heat transfer system 115 previously described with reference to others figures are shown schematically.

FIG. 15D illustrates another system 1598 which is generally similar to the system 1590 shown and described with reference to FIGS. 15B and 15C, but the package 1550 and its components (e.g., the heat exchanger 1560, pump 1562, power source 1592, and/or controller 1594) are embedded within the device 1500, e.g., physically above the TECs or majority of device components.

The systems 1590, 1598 described with reference to respective FIGS. 15C and 15D are shown to be operably coupled to a single device. In some embodiments, the system 1590 or system 1598 can be operably coupled to multiple devices, e.g., on or around different target areas of the human 10. For example, in some embodiments the system 1590 (or system 1598) can be operatively coupled to a first device around the knee region and a second device over the shoulder region. In such embodiments, the system 1590 (or system 1598) can individually control the first device independent (and individual TECs 110 thereon) from the second device, or vice versa.

Each of the devices shown in FIGS. 15A-19 can be used to treat a number of underlying conditions experienced at the target area, such as pain, swelling, overheating (e.g., for cancer patients), diminished blood perfusion, diminished nerve connectivity, and/or stroke, amongst other conditions. Moreover, each of the devices shown in FIGS. 15A-19 can be designed based on the particular area of treatment. That is, in addition to designing the device to conform to the geometry of the target area, as shown in FIGS. 15A-19, other characteristics (e.g., thickness, flexibility, density of TECs, compressive force applied to the target area, etc.) may be incorporated into the design based on the target area. For example, the device 1500 around the knee region of the human 10 can be designed to have increased flexibility at the knee joint area of the device 1500 expected to experience the most bending, and thus may include fewer TECs adjacent that area. In some embodiments, the design may be based on the expected treatment to be provided via the particular device. For example, the devices 1800 and 1900 around the head regions of the human 10 can be particularly useful for treating patients that have experienced a stroke and that need relatively quick cooling of the head region following the stroke event (e.g., in the ambulance or at the hospital). Accordingly, the devices 1800 and 1900 may be preprogrammed with an operating mode configured to thermally treat a patient that has recently experienced a stroke or other relevant condition.

Any one of the heat transfer devices 100, 700, 800, 800, 900, 1000, 1100, 1200, 1300 1400, 1500, 1600, 1700, 1800, 1900, 2000, 2100, 2200, 2300, or 2400 described herein with reference to FIGS. 1-24 can be incorporated into a myriad of other and/or more complex systems, a representative example of which is system 2590 shown schematically in FIG. 25. The system 2590 can include a heat transfer device (e.g., the heat transfer device 100, 700, 800, 800, 900, 1000, 1100, 1200, 1300 1400, 1500, 1600, 1700, 1800, 1900, 2000, 2100, 2200, 2300, or 2400), a power source 2592 (e.g., a portable power source, battery, etc.) operatively coupled to the device (e.g., to the TECs of the device), a controller 2594 (e.g., a processor) operatively coupled to the device and the power source 2592, a user interface 2596 operatively coupled to the controller 2594 and the power source 2592, as well as other subsystems. The system 2590 can perform any of a wide variety of functions, such as memory storage, data processing, and/or other suitable functions.

The controller 2594 can be configured to operate the device in one of the operating modes (e.g., a cooling mode, a heating mode, or both), and/or provide a process value (e.g., a set temperature) at which the device is configured to operate. As previously described with reference to FIG. 1 for example, the controller 2594 can provide a setpoint temperature within a range of 40° C. to −20° C. (e.g., 35° C., 20° C., 0° C., −10° C., etc.) to the device such that the TECs 110 (e.g., the first or second side of the TECs) are configured to operate at the setpoint temperature. Additionally or alternatively, the controller 2594 can be configured to receive inputs from sensors (e.g., sensors 180a-f; FIG. 4) on the device and control the device based on the received inputs. For example, the controller 2594 can determine any abnormalities of the operating device and automatically generate indications of the abnormalities and/or adjust the operating parameters of the device. Additionally or alternatively, the controller 2594 may utilize artificial intelligence and/or machine learning to adjust power and/or other control parameters, e.g., based on previous treatments used for the same user or a group of users.

The user interface 2596 can include a display, and/or an application or program that enables the user to utilize the device through a mobile device (e.g., a phone, tablet, watch, laptop, etc.) or other computing device. The user interface 2596 may include pre-programmed thermal management procedures and/or enable the user to adjust cooling and heating parameters based on a desired application.

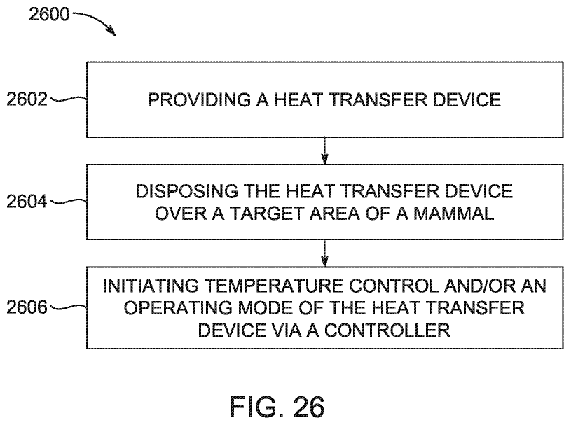

FIG. 26 is a flow diagram illustrating a method 2600 for treating a human (e.g., for pain, swelling, overheating, diminished blood perfusion, diminished nerve connectivity, and/or stroke) via a heat transfer device, in accordance with embodiments of the present technology. The method 2600 can comprise providing a heat transfer device (e.g., the heat transfer device 100, 700, 800, 800, 900, 1000, 1100, 1200, 1300 1400, 1500, 1600, 1700, 1800, 1900, 2000, 2100, 2200, 2300, or 2400) (process portion 2602), and disposing the heat transfer device over a target area of a human (process portion 2604). Disposing the heat transfer device over the target area can comprise fastening the device over the target area, e.g., such that the device or flexible support unit of the device provides a compressive force on the target area and positions TECs of the device in thermal contact with the target area.

The method 2600 can further comprise initiating temperature control and/or an operating mode of the heat transfer device via a controller (e.g., the controller 2594; FIG. 25), thereby causing heat to transfer from the target area of the human to the heat transfer device or vice versa (process portion 2606). Initiating the operating mode can include initiating a cooling mode, a heating mode, or both a cooling mode and a heating mode. Initiating the temperature control can comprise providing a temperature for the TECs (e.g., the TECs 110; FIGS. 1-6) to operate at or a temperature at which the device is configured to heat or cool the target area within a predetermined time (e.g., 10 seconds, 20 seconds, 30 seconds, 40 seconds, 60 seconds, or 120 seconds). In some embodiments, the temperature can be set to be within a range of 40° C. to −20° C. (e.g., 35° C., 20° C., 0° C., −10° C., etc.).

FIG. 27 is a flow diagram illustrating a method 2700 for controlling the temperature of a target area of a human via a heat transfer device, in accordance with embodiment of the present technology. The method 2700 can comprise calculating an incoming heat flux of the heat transfer device (e.g., the heat transfer device 100, 700, 800, 800, 900, 1000, 1100, 1200, 1300 1400, 1500, 1600, 1700, 1800, 1900, 2000, 2100, 2200, 2300, or 2400) or individual or groups of modules (e.g., modules 110, 710, 810, 910, 1010, 1110, 1210, 1310, or modules within the groups 1460-1464), given a current power input into the heat transfer device or module or modules, a heated working fluid temperature at the heat transfer device or module or modules, and a cold working fluid temperature at the heat transfer device or module or modules (process portion 2702); calculating a temperature of the target area of the human, given the income heat flux and properties of a flexible support unit proximate to the heat transfer device or module or modules (process portion 2704); and determining a modified (or maintained) power input into the heat transfer device or module or modules, given the temperature of the target area of the human and a target temperature for the target area of the human (process portion 2706). Depending on whether the desired outcome is achieved at the target area, this process may be repeated.

### IV. Conclusion

It will be apparent to those having skill in the art that changes may be made to the details of the above-described embodiments without departing from the underlying principles of the present disclosure. In some cases, well known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Throughout this disclosure, the singular terms “a,” “an,” and “the” include plural referents unless the context clearly indicates otherwise. The term “and/or” when used in reference to a list of two or more item is to be interpreted as including (a) any single item in the list, (b) all of the items in the list, or (c) any combination of the items in the list. Additionally, the term “comprising,” “including,” and “having” should be interpreted to mean including at least the recited feature(s) such that any greater number of the same feature and/or additional types of other features are not precluded.

Reference herein to “one embodiment,” “an embodiment,” “some embodiments” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims, are to be understood as being modified in all instances by the term “about” or “approximately.” The terms “about” or “approximately” when used in reference to a value are to be interpreted to mean within 10% of the stated value. Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

The disclosure set forth above is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples may be combined in any combination, and placed into a respective independent example. The other examples can be presented in a similar manner.

1. A thermal management device, comprising:
thermoelectric components arranged in an array and spaced apart from each other, wherein individual thermoelectric components have a first side configured to be thermally coupled to a target area of a mammal and a second side opposite the first side; and
a heat transfer system having a heat exchanger, an array of fluid distribution networks, an inlet passage fluidically coupled to the fluid distribution networks and configured to provide a working fluid to the fluid distribution networks, and an outlet passage fluidically coupled to fluid distribution networks and configured to receive the working fluid from the fluid distribution networks, wherein individual fluid distribution networks are thermally coupled to the second side of a corresponding one of the thermoelectric components and fluidically coupled to the heat exchanger via the inlet passage and the outlet passage, wherein each of the fluid distribution networks has an inlet region and an outlet region, and wherein, in operation, the working fluid flows from the inlet region through the fluid distribution networks to the outlet region.

2. The thermal management device of any one of the clauses herein, wherein
the fluid distribution networks include a first fluid distribution network and a second fluid distribution network,
the first fluid distribution network is coupled to a first area of the inlet passage and the second fluid distribution network is coupled to a second area of the inlet passage downstream of the first area of the inlet passage, and
the first fluid distribution network is coupled to a first area of the outlet passage and the second fluid distribution network is coupled to a second area of the outlet passage downstream of the first area of the outlet passage.

3. The thermal management device of clause 2, wherein, in operation, a difference in pressure of the working fluid measured between the first area of the inlet passage and the first area of the outlet passage is approximately equal to a difference in pressure of the working fluid measured between the second area of the inlet passage and the second area of the outlet passage.

4. The thermal management device of clause 2, wherein, in operation:
the working fluid flowing between the first area of the inlet passage and the first area of the outlet passage has a first pressure drop,
the working fluid flowing between the second area of the inlet passage and the second area of the outlet passage has a second pressure drop, and
a difference between the first pressure drop and the second pressure drop is the same or within 5%, 10%, or 15% of one another.

5. The thermal management device of clause 2, wherein, in operation:
the working fluid flowing between the first area of the inlet passage and the first area of the outlet passage has a first pressure drop,
the working fluid flowing between the second area of the inlet passage and the second area of the outlet passage has a second pressure drop, and
a difference between the first pressure drop and the second pressure drop is less than a predetermined threshold.

6. The thermal management device of clause 2, wherein, in operation the working fluid at the first area of the inlet passage and the working fluid at the second area of the inlet passage has the same temperature.

7. The thermal management device of clause 2, wherein, in operation the working fluid at the first area of the inlet passage has a first temperature and the working fluid at the second area of the inlet passage has a second temperature, a difference between the first temperature and the second temperature being less than a predetermined threshold of 1° C., 2° C., 3° C., 4° C., or 5° C.

8. The thermal management device of clause 2, wherein the first area of the outlet passage is a proximal terminus of the outlet passage.

9. The thermal management device of clause 2, wherein the second area of the inlet passage is a distal terminus of the inlet passage.

10. The thermal management device of clause 2, wherein the heat transfer system is a closed loop system.

11. The thermal management device of clause 2, wherein the inlet passage is a cold working fluid passage configured to direct cooled working fluid from the heat exchanger to the fluid distribution networks, and the outlet passage is a hot working fluid passage configured to direct heated working fluid from the fluid distribution networks to the heat exchanger.

12. The thermal management device of clause 2, wherein the fluid distribution networks further include a third fluid distribution network, wherein the third fluid distribution network is coupled to (i) a third area of the inlet passage downstream of the second area of the inlet passage and (ii) a third area of the outlet passage downstream of the second area of the outlet passage.

13. The thermal management device of clause 12, wherein, in operation, a difference in pressure of the working fluid measured between the first area of the inlet passage and the first area of the outlet passage is approximately equal to: (i) a difference in pressure of the working fluid measured between the second area of the inlet passage and the second area of the outlet passage and (ii) a difference in pressure of the working fluid measured between the third area of the inlet passage and the third area of the outlet passage.

14. The thermal management device of clause 12, wherein, in operation:
the working fluid flowing between the first area of the inlet passage and the first area of the outlet passage experiences a first pressure drop,
the working fluid flowing between the second area of the inlet passage and the second area of the outlet passage experiences a second pressure drop,
the working fluid flowing between the third area of the inlet passage and the third area of the outlet passage experiences a third pressure drop, and
a difference between the first pressure drop, the second pressure drop, and the third pressure drop is the same or within 5%, 10%, or 15% of one another.

15. The thermal management device of clause 2, wherein, in operation:
the working fluid flowing between the first area of the inlet passage and the first area of the outlet passage has a first pressure drop,
the working fluid flowing between the second area of the inlet passage and the second area of the outlet passage has a second pressure drop, and
a difference between the first pressure drop and the second pressure drop is less than a predetermined threshold.

16. The thermal management device of any one of the clauses herein, wherein each of the fluid distribution networks includes microfeatures spaced apart from each other to at least partially define channels configured to receive the working fluid.

17. The thermal management device of any one of the clauses herein, further comprising a flexible support member including a first side and a second side opposite the first side, wherein the first side of the flexible support member is coupled to the first side of the thermoelectric components and the second side of the flexible support member is configured to be disposed on the target area of the mammal.

18. The thermal management device of clause 17, wherein the flexible support member is expandable along at least one of a first dimension or a second dimension normal to the first dimension.

19. The thermal management device of clause 17, further comprising one or more thermally conductive members extending along all or part of a dimension of the flexible support member.

20. The thermal management device of clause 17, wherein the thermally conductive members comprise copper, brass, steel, zinc, or alloys thereof.

21. The thermal management device of clause 17, wherein the thermally conductive member has a rigidity higher than that of the flexible support member.

22. The thermal management device of clause 17, wherein the thermally conductive members comprise a conductive wire, a stamped conductive metal, or a sheet of thermally conductive material.

23. The thermal management device of clause 19, wherein the thermally conductive members have an oscillating or wave-like shape.

24. The thermal management device of clause 19, wherein the thermally conductive members include a first thermally conductive member extending in a first direction and a second thermally conductive member extending in a second direction normal to the first direction.

25. A thermal management device, comprising:
thermoelectric components arranged in an array and spaced apart from each other, wherein individual thermoelectric components have a first side configured to be thermally coupled to a target area of a mammal and a second side opposite the first side;
a heat transfer system having a heat exchanger, an array of fluid distribution networks, an inlet passage fluidically coupled to the fluid distribution networks and configured to provide a working fluid to the fluid distribution networks, and an outlet passage fluidically coupled to fluid distribution networks and configured to receive the working fluid from the fluid distribution networks, wherein individual fluid distribution networks are thermally coupled to the second side of a corresponding one of the thermoelectric components and fluidically coupled to the heat exchanger via the inlet passage and the outlet passage, wherein each of the fluid distribution networks has an inlet region, an outlet region, and microfeatures spaced apart from each other to at least partially define channels configured to receive the working fluid, wherein, in an operation mode, the working fluid flows from the inlet region to the outlet region and absorbs heat from the microfeatures,
the fluid distribution networks including a first fluid distribution network and a second fluid distribution network, wherein
   the inlet passage is positioned to provide the working fluid to the first fluid distribution network at a first temperature and first pressure and the second fluid distribution network at a second temperature and second pressure,
   the first temperature and the second temperature are about equal, and
   the first pressure is greater than the second pressure.

26. The thermal management device of any one of the clauses herein, wherein a first pressure drop between the inlet region and the outlet region of the first fluid distribution network and a second pressure drop between the inlet region and the outlet region of the second fluid distribution network are equal.

27. The thermal management device of any one of the clauses herein, wherein the first fluid distribution network has a first working fluid flow rate and the second fluid distribution network has a second working fluid flow rate, wherein the first and the second working fluid flow rates are equal.

28. The thermal management device of any one of the clauses herein, wherein the heat exchanger provides a cold working fluid to the fluid distribution networks and receives a heat working fluid from the fluid distribution networks.

29. The thermal management device of any one of the clauses herein, wherein the operation mode is a first operation mode, the thermal management device further comprising a second operation mode wherein the working fluid provides heat to the microfeatures.

30. A thermal management device, comprising:
thermoelectric components arranged in an array and spaced apart from each other, wherein individual thermoelectric components have a first side configured to be thermally coupled to a target area of a mammal and a second side opposite the first side;
a heat transfer system having a heat exchanger and an array of fluid distribution networks in which individual fluid distribution networks are thermally coupled to the second side of a corresponding one of the thermoelectric components and fluidically coupled to the heat exchanger, wherein each of the fluid distribution networks has an inlet region, an outlet region, and microfeatures spaced apart from each other to at least partially define channels configured to receive a working fluid, wherein, in operation, the working fluid flows from the inlet region to the outlet region and absorbs heat from the microfeatures;
a flexible support unit coupled to the thermoelectric components and configured such that, when attached to the mammal, the thermoelectric components are arranged to be adjacent to the target area, wherein the flexible support unit is configured to exert a compressive force against the target area; and
a thermally conductive member coupled to the flexible support unit and in thermal communication along a dimension of the flexible support unit across two or more of the thermoelectric components.

31. The thermal management device of any one of the clauses herein, wherein the thermally conductive member is a conductive wire in a waved pattern across two or more of the thermoelectric components.

32. The thermal management device of any one of the clauses herein, wherein the thermally conductive member is a stamped conductive metal in a waved pattern across two or more of the thermoelectric components.

33. The thermal management device of any one of the clauses herein, wherein the thermally conductive member is a sheet of thermally conductive material across two of more of the thermoelectric components.

34. The thermal management device of any one of the clauses herein, wherein the sheet of thermally conductive material defines a cutout, wherein the cutout is aligned with at least one of the thermoelectric components.

35. The thermal management device of any one of the clauses herein, wherein the thermally conductive member is one of several thermally conductive members.

36. The thermal management device of clause 35, wherein a first set of the several thermally conductive members substantially aligns with the dimension of the flexible support unit, and a second set of the several thermally conductive members is misaligned from the dimension of the flexible support unit.

37. The thermal management device of clause 35, wherein the several thermally conductive members have a higher rigidity than the flexible support unit.

38. The thermal management device of clause 35, wherein the several thermally conductive members are configured like springs within the flexible support unit, such that when the flexible support unit and the several thermally conductive members are expanded along the dimension of the flexible support unit, the several thermally conductive members exert a spring-biasing force on the flexible support unit.

39. The thermal management device of clause 35, wherein the several thermally conductive members are conductive particles within the flexible support unit.

40. The thermal management device of any one of the clauses herein, wherein the thermally conductive member is embedded within the flexible support unit.

41. A thermal management device, comprising:
thermoelectric components arranged in an array and spaced apart from each other, wherein individual thermoelectric components have a first side configured to be thermally coupled to a target area of a mammal and a second side opposite the first side;
a heat transfer system having a heat exchanger and an array of fluid distribution networks in which individual fluid distribution networks are thermally coupled to the second side of a corresponding one of the thermoelectric components and fluidically coupled to the heat exchanger, wherein each of the fluid distribution networks has an inlet region, an outlet region, and microfeatures spaced apart from each other to at least partially define channels configured to receive a working fluid, wherein, in operation, the working fluid flows from the inlet region to the outlet region and absorbs heat from the microfeatures;
a flexible support unit having an effective thermal conductivity and being coupled to the thermoelectric components and configured such that, when attached to the mammal, the thermoelectric components are arranged to be adjacent to the target area, wherein the flexible support unit is configured to exert a compressive force against the target area; and
a controller coupled to the thermoelectric components, wherein the controller is configured to collect from the thermoelectric components several temperature readings at the first sides and second sides, evaluate the collected temperature readings in reference to the effective thermal conductivity of the flexible support unit to identify a temperature of the target area, and modify an input to the thermoelectric components such that the thermoelectric components changes the temperature of the target area to a target temperature within a predetermined period of time.

42. The thermal management device of any one of the clauses herein, wherein the flexible support unit comprises a thermally conductive flexible member coupled to the first sides of the thermoelectric components and extending at least between individual thermoelectric components, wherein the thermoelectric components are thermally coupled to the target area via the thermally conductive flexible member.

43. The thermal management device of any one of the clauses herein, further comprising a first and a second thermoelectric component group, the first and the second thermoelectric component groups each including at least two thermoelectric components and at least two fluid distribution networks.

44. The thermal management device of clause 43, wherein the controller is further configured to independently collect from the first and the second thermoelectric component groups temperature readings at the first sides and second sides, evaluate the collected temperature readings in reference to the effective thermal conductivity of the flexible support unit to identify the temperature of the target area, and modify the input to the thermoelectric component groups such that the thermoelectric component groups independently change the temperature of a target area proximate the first thermoelectric component group to a first target temperature within a first predetermined period of time and a target area proximate the second thermoelectric component group to a second target temperature within a second predetermined period of time.

45. The thermal management device of clause 44, wherein the first and second predetermined period of time are the same.

46. A method for controlling a temperature of a target area of a mammal within a predetermined period of time, comprising:
providing a wearable heat transfer device including
   thermoelectric components each having a first side and a second side opposite the first side;
   an array of fluid distribution networks each being thermally coupled to the second side of a corresponding one of the thermoelectric components and fluidically coupled to a heat exchanger, wherein each of the fluid distribution networks has an inlet region fluidically coupled to a common inlet fluid distribution passage and an outlet region fluidically coupled to a common outlet fluid distribution passage, wherein, in operation, a working fluid disposed within the fluid distribution network is configured to absorb heat from the corresponding one of the thermoelectric components; and
   a flexible support unit coupled to the first sides of the thermoelectric components and extending at least between individual thermoelectric components, the flexible support unit being a heat spreader configured to enhance heat transfer from the mammal;
disposing the heat transfer device over the target area of the mammal such that the thermoelectric components of the heat transfer device are thermally coupled to the target area; and
initiating, via a controller operatively coupled to the heat transfer device, temperature control of the heat transfer device, including
   collecting several temperature readings at the first sides and the second sides of the thermoelectric components using the controller,
   evaluating the collected several temperature readings in reference to an effective thermal conductivity of the flexible support unit to identify the temperature of the target area of the mammal,
   modifying an input to the thermoelectric components such that the thermoelectric components change the temperature of the target area toward a desired temperature, and
   repeating collecting, evaluating, and modifying until the temperature of the target area is equal to the desired temperature.

47. The method of any one of the clauses herein, wherein the flexible support unit comprises a thermally conductive flexible member coupled to the first sides of the thermoelectric components and extending at least between individual thermoelectric components, wherein the thermoelectric components are thermally coupled to the target area via the thermally conductive flexible member.

48. The method of clause 47, wherein disposing the heat transfer device over the target area comprises disposing the thermally conductive flexible member directly against the mammal.

49. The method of any one of the clauses herein, further comprising a first and a second thermoelectric component group, the first and the second thermoelectric component groups each including at least two thermoelectric components and at least two fluid distribution networks.

50. The method of clause 49, wherein the controller is further configured to independently collect from the first and the second thermoelectric component groups the temperature reading at the first sides and second sides, evaluate the collected temperature readings in reference to the effective thermal conductivity of the flexible support unit to identify the temperature of the target area, and modify the input to the thermoelectric component groups such that the thermoelectric component groups independently change the temperature of a target area proximate the first thermoelectric component group to a first target temperature within a first predetermined period of time and a target area proximate the second thermoelectric component group to a second target temperature within a second predetermined period of time.

51. The method of clause 50, wherein the first and second predetermined period of time are the same.

I/We claim:

1. A thermal management device, comprising:
thermoelectric components arranged in an array and spaced apart from each other, wherein individual thermoelectric components have a first side configured to be thermally coupled to a target area of a mammal and a second side opposite the first side; and
a heat transfer system including:
a heat exchanger,
an array of fluid distribution networks,
an inlet passage fluidically coupled to the fluid distribution networks and configured to provide a working fluid to the fluid distribution networks, and
an outlet passage fluidically coupled to fluid distribution networks and configured to receive the working fluid from the fluid distribution networks,
wherein:
individual fluid distribution networks are thermally coupled to the second side of a corresponding one of the thermoelectric components and fluidically coupled to the heat exchanger via the inlet passage and the outlet passage,
the individual fluid distribution networks have an inlet region and an outlet region, and, in operation, the working fluid flows from the inlet region through the fluid distribution networks to the outlet region,
the fluid distribution networks include a first fluid distribution network and a second fluid distribution network,
the first fluid distribution network is coupled to a first area of the inlet passage and the second fluid distribution network is coupled to a second area of the inlet passage downstream of the first area of the inlet passage, and
the first fluid distribution network is coupled to a first area of the outlet passage and the second fluid distribution network is coupled to a second area of the outlet passage downstream of the first area of the outlet passage.

2. The thermal management device of claim 1, wherein, in operation, a difference in pressure of the working fluid measured between the first area of the inlet passage and the first area of the outlet passage is approximately equal to a difference in pressure of the working fluid measured between the second area of the inlet passage and the second area of the outlet passage.

3. The thermal management device of claim 1, wherein, in operation:
the working fluid flowing between the first area of the inlet passage and the first area of the outlet passage has a first pressure drop,
the working fluid flowing between the second area of the inlet passage and the second area of the outlet passage has a second pressure drop, and
a difference between the first pressure drop and the second pressure drop is within 10% of one another.

4. The thermal management device of claim 1, wherein, in operation, the working fluid has:
a first pressure at the first area of the inlet passage,
a second pressure, less than the first pressure, at the first area of the outlet passage,
a third pressure, less than the first pressure, at the second area of the inlet passage, and
a fourth pressure, less than the third pressure, at the second area of the outlet passage, and
a difference between the first pressure drop and the second pressure drop is less than a predetermined threshold.

5. The thermal management device of claim 1, wherein, in operation the working fluid at the first area of the inlet passage and the working fluid at the second area of the inlet passage has the same temperature.

6. The thermal management device of claim 1, wherein, in operation the working fluid at the first area of the outlet passage has a first temperature and the working fluid at the second area of the outlet passage has a second temperature, and wherein a difference between the first temperature and the second temperature is approximately the same or less than a predetermined threshold.

7. The thermal management device of claim 1, wherein the first area of the outlet passage is a proximal terminus of the outlet passage.

8. The thermal management device of claim 7, wherein the second area of the inlet passage is a distal terminus of the inlet passage.

9. The thermal management device of claim 1, wherein the heat transfer system is a closed loop system.

10. The thermal management device of claim 1, wherein the inlet passage is a cold working fluid passage configured to direct cooled working fluid from the heat exchanger to the fluid distribution networks, and the outlet passage is a hot working fluid passage configured to direct heated working fluid from the fluid distribution networks to the heat exchanger.

11. The thermal management device of claim 1, wherein the fluid distribution networks further include a third fluid distribution network, wherein the third fluid distribution network is coupled to (i) a third area of the inlet passage downstream of the second area of the inlet passage and (ii) a third area of the outlet passage downstream of the second area of the outlet passage.

12. The thermal management device of claim 11, wherein, in operation, a difference in pressure of the working fluid measured between the first area of the inlet passage and the first area of the outlet passage is approximately equal to: (i) a difference in pressure of the working fluid measured between the second area of the inlet passage and the second area of the outlet passage and (ii) a difference in pressure of the working fluid measured between the third area of the inlet passage and the third area of the outlet passage.

13. The thermal management device of claim 11, wherein, in operation, the working fluid has:
a first pressure at the first area of the inlet passage,
a second pressure, less than the first pressure, at the first area of the outlet passage,
a third pressure, less than the first pressure, at the second area of the inlet passage,
a fourth pressure, less than the third pressure, at the second area of the outlet passage,
a fifth pressure, less than the second pressure, at the third area of the inlet passage, and
a sixth pressure, less than the fifth pressure, at the third area of the outlet passage.

14. The thermal management device of claim 13, wherein, in operation:
the working fluid flowing between the first area of the inlet passage and the first area of the outlet passage has a first pressure drop,
the working fluid flowing between the second area of the inlet passage and the second area of the outlet passage has a second pressure drop,
the working fluid flowing between the third area of the inlet passage and the third area of the outlet passage has a third pressure drop, and
a difference between (i) the first pressure drop and the second pressure drop and (ii) the second pressure drop and the third pressure drop is approximately equal.

15. A thermal management device, comprising:
thermoelectric components arranged in an array and spaced apart from each other, wherein individual thermoelectric components have a first side configured to be thermally coupled to a target area of a mammal and a second side opposite the first side; and
a heat transfer system including:
a heat exchanger,
an array of fluid distribution networks,
an inlet passage fluidically coupled to the fluid distribution networks and configured to provide a working fluid to the fluid distribution networks, and
an outlet passage fluidically coupled to at least some of the fluid distribution networks and configured to receive the working fluid from the fluid distribution networks, wherein:
individual fluid distribution networks are thermally coupled to the second side of a corresponding one of the thermoelectric components and fluidically coupled to the heat exchanger via the inlet passage and the outlet passage,
each of the fluid distribution networks has an inlet region, an outlet region, and microfeatures spaced apart from each other to at least partially define channels configured to receive the working fluid,
in an operation mode, the working fluid flows from the inlet region to the outlet region and absorbs heat from the microfeatures,
the fluid distribution networks including a first fluid distribution network and a second fluid distribution network, wherein
the inlet passage is positioned to provide the working fluid to (i) the first fluid distribution network at a first temperature and first pressure and (ii) the second fluid distribution network at a second temperature and second pressure,
the first temperature and the second temperature are equal, and
the first pressure is greater than the second pressure.

16. The thermal management device of claim 15, wherein a first pressure drop between the inlet region and the outlet region of the first fluid distribution network and a second pressure drop between the inlet region and the outlet region of the second fluid distribution network are equal.

17. The thermal management device of claim 15, wherein the first fluid distribution network has a first working fluid flow rate and the second fluid distribution network has a second working fluid flow rate, wherein the first and the second working fluid flow rates are equal.

18. The thermal management device of claim 15, wherein the heat exchanger provides a cold working fluid to the fluid distribution networks and receives a heat working fluid from the fluid distribution networks.

19. The thermal management device of claim 15, wherein the operation mode is a first operation mode, the thermal management device further comprising a second operation mode wherein the working fluid provides heat to the microfeatures.

20. A thermal management device, comprising:
thermoelectric components arranged in an array and spaced apart from each other, wherein individual thermoelectric components have a first side configured to be thermally coupled to a target area of a mammal and a second side opposite the first side;
a heat transfer system having a heat exchanger and an array of fluid distribution networks in which individual fluid distribution networks are thermally coupled to the second side of a corresponding one of the thermoelectric components and fluidically coupled to the heat exchanger, wherein each of the fluid distribution networks has an inlet region, an outlet region, and microfeatures spaced apart from each other to at least partially define channels configured to receive a working fluid, wherein, in operation, the working fluid flows from the inlet region to the outlet region and absorbs heat from the microfeatures;
a flexible support unit coupled to the thermoelectric components and configured such that, when attached to the mammal, the thermoelectric components are arranged to be adjacent to the target area, wherein the flexible support unit is configured to exert a compressive force against the target area; and
a thermally conductive member coupled to the flexible support unit and in thermal communication along a dimension of the flexible support unit across two or more of the thermoelectric components.

21. The thermal management device of claim 20, wherein the thermally conductive member is a conductive wire in a waved pattern across two or more of the thermoelectric components.

22. The thermal management device of claim 20, wherein the thermally conductive member is a sheet of thermally conductive material across two of more of the thermoelectric components.

23. The thermal management device of claim 22, wherein a first set of the several thermally conductive members substantially aligns with the dimension of the flexible support unit, and a second set of the several thermally conductive members is misaligned from the dimension of the flexible support unit.

24. The thermal management device of claim 22, wherein the several thermally conductive members have a higher rigidity than the flexible support unit.

25. The thermal management device of claim 22, wherein the several thermally conductive members are configured like springs within the flexible support unit, such that when the flexible support unit and the several thermally conductive members are expanded along the dimension of the flexible support unit, the several thermally conductive members exert a spring-biasing force on the flexible support unit.

26. The thermal management device of claim 20, wherein the thermally conductive member is embedded within the flexible support unit.

27. A method for controlling a temperature of a target area of a mammal within a predetermined period of time, comprising:
providing a wearable heat transfer device including
thermoelectric components each having a first side and a second side opposite the first side;
an array of fluid distribution networks each being thermally coupled to the second side of a corresponding one of the thermoelectric components and fluidically coupled to a heat exchanger, wherein each of the fluid distribution networks has an inlet region fluidically coupled to a common inlet fluid distribution passage and an outlet region fluidically coupled to a common outlet fluid distribution passage, wherein, in operation, a working fluid within the fluid distribution network is configured to absorb heat from the corresponding one of the thermoelectric components; and
a flexible support unit coupled to the first sides of the thermoelectric components and extending at least between individual thermoelectric components, the flexible support unit being a heat spreader configured to enhance heat transfer from the mammal;
disposing the heat transfer device over the target area of the mammal such that the thermoelectric components of the heat transfer device are thermally coupled to the target area; and
initiating, via a controller operatively coupled to the heat transfer device, temperature control of the heat transfer device, including
collecting temperature readings at the first sides and the second sides of the thermoelectric components using the controller,
evaluating the collected temperature readings in reference to an effective thermal conductivity of the flexible support unit to identify the temperature of the target area of the mammal, and
modifying an input to at least some of the thermoelectric components such that the at least some of the thermoelectric components change the temperature of the target area toward a desired temperature.

28. The method of claim 27, wherein the flexible support unit comprises a thermally conductive flexible member coupled to the first sides of the thermoelectric components and extending at least between individual thermoelectric components, wherein the thermoelectric components are thermally coupled to the target area via the thermally conductive flexible member.

29. The method of claim 27, further comprising a first thermoelectric component group and a second thermoelectric component group, the first and the second thermoelectric component groups each including at least two thermoelectric components and at least two fluid distribution networks, wherein modifying the input comprises modifying a first input provided to the first thermoelectric components group and modifying a second input provided to the second thermoelectric component group.

30. The method of claim 27, further comprising iteratively repeating the collecting, evaluating, and modifying steps at least twice until the temperature of the target area is equal to the desired temperature.
